SEQUENCE LISTING (17 sequences)

SEQ ID NO: 1 - DNA, 2751 bp, Arabidopsis thaliana
atggctctaaagtttaaccctttggtggcatctcagccttacaaattcccttcctcgactcgtccgccaactccttctttcagatctcccaagttcctctgcctcgcttcttcttctccggctctcagctccggccccaagtcagtctctttctctccatcatcgtctctcttatctactttcgattctctctgtttcatctttgtttactgtgttctgggttttgcttgttcttatcgatattggatctacgttttctcgattttgttcagtgttatttgcttacatttgcttaatctcgagtgtgatttcaataaaagtacgttcttttaatctgggtttataagaatgggccatcctctgttctcaggtgattcattaatgctctttttcattgaatctgatgtttgttgattgttccctattattctcacgtttctctgagattgccccatctatcaaatcgttgcctttaccatatttattctgcatcagattatgtagattgctcgttgattggttgttagttaagattaacactagttatcttctttatggtataatgatctttagattggttaagacttaacagacattgaggaaagcttcttagtcaaagagaaagttttgttttgttttctctggtttagtaatttttaatggtgaaatgtttatccacttgtttcttactcatttgatgagttttctcttttgtattgacaagagattcttgaacgtatcagtttacgtctgtttatctttcttcaaagacattacgttggatatcacgcttcgtggcctttctctatgcatgcgtacttcgtggcttctctctttagtttgcgtccaaaattttcacacttttctgtaatttattattcattctttctctgttttgaagacatttggctctttatcttgtctttgtctgatcagtagtcactacatttgtttctttcacaatctttcttttttgcttcatattgaactgcaagaccaatccatgaatgcaaatataaaatattgtcttacggttaaccatctctaagtgtttgctagtcatgaattgtgaaattgtttcgcttcaaacatagaattagttgatttggaaatgtagacatgaatgcttatctaaatacaataaactgtgttcttgtttcagttgtgtatcagagatgagaccttgactggtaatttcaatggagcataattaaatgctaatgatacttattttctggttaatgcagggaggttgagagtttgaagaaaccatttacgccacccagggaagtgcatgttcaagtcttgcactccatgccacctcaaaagatcgagatcttcaaatctatggaaaactgggccgaggagaaccttctgattcacctcaaggatgtggagaagtcttggcaaccccaggatttcttgcctgaccctgcatcagatgggtttgaagatcaggtaagagagttaagagagagggctagagagctccctgatgattactttgttgttttggtgggggacatgatcacagaagaagcacttccgacctatcaaactatgttgaacactttggatggagttagggatgaaacaggtgctagtcctacttcatgggctatttggaccagagcttggactgcagaagaaaaccgacatggcgatcttctgaataaatacctttacttgtctggtcgtgttgacatgaggcagatcgaaaagaccattcagtacttgattggatctggaatggtgagatagtttcaggcaattatcatgatttcttggttaatataactacctaatcgctttaacttatctttcataggatccgcggacagagaataacccctaccttggcttcatctatacgtcattccaagaaagagcgacattcatctctcacggaaacacagcccgccaagccaaagagcacggggacatcaaactagcccaaatatgtggcacaatagctgcagacgagaagcgtcatgaaacagcatacaccaagatagttgaaaagctctttgagattgatcctgatggtactgtcatggcttttgcagacatgatgagaaagaaaatctcaatgcctgctcacttgatgtatgatgggcgcaacgacaacctctttgacaacttctcttccgtggctcagaggctcggtgtttacaccgccaaagactatgcagacattcttgagtttctggttggtaggtggaaaatccaggacttaaccgggctttcaggtgaaggaaacaaagcacaagactatttatgcgggttggctccaaggatcaagagattggatgagagagctcaagcaagagccaagaaaggacccaagattcctttcagttggatacacgacagagaagtgcagctctaaaaggacaaagacaaaaacaaaaacctatcctcccggttcctcatttcatctgtctgctcttaaaattggtgtagattactatggttttctgataatgttggtgggtctagttacaaagttgagatgcagtgatttagtagctttgtttttcccagtcactatatgtttggtctttggtccgttagcacacttgtagtagttaaaacagtttaagtatggtctgtactcagtcttcctcttctctgtggagttttgtttaagttcaggttagttttgttttgtctctctgttttttcccctgtttctcgacaaacaactcatgtggctttttagcaattttgatggatgatcatgatgaataa

SEQ ID NO: 2 - DNA, 1206 bp, Arabidopsis thaliana
atggctctaaagtttaaccctttggtggcatctcagccttacaaattcccttcctcgactcgtccgccaactccttctttcagatctcccaagttcctctgcctcgcttcttcttctccggctctcagctccggccccaaggaggttgagagtttgaagaaaccatttacgccacccagggaagtgcatgttcaagtcttgcactccatgccacctcaaaagatcgagatcttcaaatctatggaaaactgggccgaggagaaccttctgattcacctcaaggatgtggagaagtcttggcaaccccaggatttcttgcctgaccctgcatcagatgggtttgaagatcaggtaagagagttaagagagagggctagagagctccctgatgattactttgttgttttggtgggggacatgatcacagaagaagcacttccgacctatcaaactatgttgaacactttggatggagttagggatgaaacaggtgctagtcctacttcatgggctatttggaccagagcttggactgcagaagaaaaccgacatggcgatcttctgaataaatacctttacttgtctggtcgtgttgacatgaggcagatcgaaaagaccattcagtacttgattggatctggaatggatccgcggacagagaataacccctaccttggcttcatctatacgtcattccaagaaagagcgacattcatctctcacggaaacacagcccgccaagccaaagagcacggggacatcaaactagcccaaatatgtggcacaatagctgcagacgagaagcgtcatgaaacagcatacaccaagatagttgaaaagctctttgagattgatcctgatggtactgtcatggcttttgcagacatgatgagaaagaaaatctcaatgcctgctcacttgatgtatgatgggcgcaacgacaacctctttgacaacttctcttccgtggctcagaggctcggtgtttacaccgccaaagactatgcagacattcttgagtttctggttggtaggtggaaaatccaggacttaaccgggctttcaggtgaaggaaacaaagcacaagactatttatgcgggttggctccaaggatcaagagattggatgagagagctcaagcaagagccaagaaaggacccaagattcctttcagttggatacacgacagagaagtgcagctctaa

SEQ ID NO: 3 - protein, 401 aa, Arabidopsis thaliana
MALKFNPLVASQPYKFPSSTRPPTPSFRSPKFLCLASSSPALSSGPKEVESLKKPFTPPREVHVQVLHSMPPQKIEIFKSMENWAEENLLIHLKDVEKSWQPQDFLPDPASDGFEDQVRELRERARELPDDYFVVLVGDMITEEALPTYQTMLNTLDGVRDETGASPTSWAIWTRAWTAEENRHGDLLNKYLYLSGRVDMRQIEKTIQYLIGSGMDPRTENNPYLGFIYTSFQERATFISHGNTARQAKEHGDIKLAQICGTIAADEKRHETAYTKIVEKLFEIDPDGTVMAFADMMRKKISMPAHLMYDGRNDNLFDNFSSVAQRLGVYTAKDYADILEFLVGRWKIQDLTGLSGEGNKAQDYLCGLAPRIKRLDERAQARAKKGPKIPFSWIHDREVQL

SEQ ID NO: 4 - protein, 60 aa, Arabidopsis thaliana
  misc_feature (26)..(26): Phe residue instead of Leu present at position corresponding to residue 146 of wild type.
LRERARELPDDYFVVLVGDMITEEAFPTYQTMLNTLDGVRDETGASPTSWAIWTRAWTAE

SEQ ID NO: 5 - protein, 60 aa, B. napus
LRERARELPDDYFVVLVGDMITEEALPTYQTMLNTLDGVRDETGASPTSWAVWTRAWTAE

SEQ ID NO: 6 - protein, 60 aa, B. juncea
LRERARELPDDYFVVLVGDMITEEALPTYQTMLNTLDGVRDETGASPTPWAVWTRAWTAE

SEQ ID NO: 7 - protein, 60 aa, Ricinis
LRERAKEIPDDYFVVLVGDMITEEALPTYQTMLNTLDGVRDETGASPTSWAIWTRAWTAE

SEQ ID NO: 8 - protein, 60 aa, Sesamum; identical to SEQ ID NO: 7

SEQ ID NO: 9 - protein, 60 aa, Glycine
LRERAKELPDDYFVVLVGDMITEEALPTYQTMLNTLDGVRDETGASLTSWAIWTRAWTAE

SEQ ID NO: 10 - protein, 60 aa, Cucumis
LRERAKELPDEYFVVLVGDMITEEALPTYQTMLNTLDGVRDETGASPTPWAIWTRAWTAE

SEQ ID NO: 11 - protein, 60 aa, Carthamus
LRARAKEIPDDYFVVLVGDMITEEALPTYQTMLNTLDGVRDETGASLTPWAVWTRAWTAE

SEQ ID NO: 12 - protein, 60 aa, Arachis
LRARAKELPDDYFVVLVGDMITEEALPTYQTMLNTLDGVRDETGASLTSWAVWTRAWTAE

SEQ ID NO: 13 - protein, 60 aa, Solanum
LRERCKEIPDDYFVVLVGDMITEEALPTYQTMLNTLDGVRDETGASLTPWAIWTRAWTAE

SEQ ID NO: 14 - protein, 60 aa, Oryza
LRERAKEIPDDYFVCLVGDMVTEEALPTYQTMLNTLDGVRDETGASPTTWAVWTRAWTAE

SEQ ID NO: 15 - protein, 52 aa, Mycobacterium
GKEQSKVTEIGRIALVVNLLTEDNLPSYHHEIASLFGRDGAWGTWVHRWTAE

SEQ ID NO: 16 - DNA, 23 bp, Artificial Sequence
  Primer
agagagggctagagagctccctg

SEQ ID NO: 17 - DNA, 29 bp, Artificial Sequence
  Primer
agtgttcaacatagtttgataggtcctaa

(19) United States
(12) Patent Application Publication
(10) Pub. No.: US 2004/0049807 A1
(43) Pub. Date: Mar. 11, 2004

(54) Fatty acid desaturase gene and protein for modulating activation of defense signaling pathways in plants

(76) Inventors: Daniel F Klessig, Ithaca, NY (US); Pradeep Kachroo, Ithaca, NY (US); Jyoti Shah, Manhattan, KS (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 10/297,813

(22) PCT Filed: May 18, 2001
(86) PCT No.: PCT/US01/16134

(51) Int. Cl. A01H 1/00; C12N 9/04; C12N 15/82; C07H 21/04; C12N 5/04

(52) U.S. Cl. 800/281; 435/69.1; 435/190; 435/320.1; 435/419; 536/23.2

(57) ABSTRACT

A novel plant gene, SSI2, is disclosed. SSI2 encodes a stearoyl-ACP desaturase in plants and plays a key role in modulating plant defense responses. Also disclosed is a FA-derived signaling molecule(s) that can be manipulated through the up- or down-regulation of the SSI2 FA desaturase, resulting in specific modifications of plant defense responses. This FA-derived signaling molecule(s) comprises at least an 18:1 FA or a derivative thereof. Mutant plants with substantially reduced SSI2 activity are also disclosed, along with transgenic plants that over- or under-express the SSI2 gene.

This application claims priority to U.S. Provisional Application No. 60/210,967, filed Jun. 12, 2000, the entirety of which is incorporated by reference herein.

Pursuant to 35 U.S.C. §202(c), it is acknowledged that the U.S. Government has certain rights in the invention described herein, which was made in part with funds from the National Science Foundation, Grant No. MCB 9723952.

### FIELD OF THE INVENTION

This invention relates to the field of plant molecular biology and plant pathology. More specifically, this invention relates to a novel pathway for defense responses in plants, and genes involved in that pathway.

### BACKGROUND OF THE INVENTION

Various publications or patents are referred to in parentheses throughout this application to describe the state of the art to which the invention pertains. Each of these publications or patents is incorporated by reference herein. Complete citations of scientific publications are set forth in the text or at the end of the specification.

The hypersensitive response (HR) and systemic acquired resistance (SAR) are important components of a plants defense arsenal against pathogens. The HR is a rapid defense response characterized by localized programmed host cell death and restriction of pathogen to the site of pathogen entry. Subsequent to the HR, a systemic signal is released that induces SAR in uninfected plant tissues. SAR is long-lasting and confers resistance against a broad spectrum of pathogens. Tightly correlated with the appearance of HR and SAR is an increase in the accumulation of salicylic acid (SA) and expression of a subset of the pathogenesis-related (PR) genes, some of which encode proteins with antimicrobial activities. Hence, the expression of these genes serves as an excellent molecular marker for a resistance response.

SA has emerged as a key signal molecule in the deployment of SAR. After the initial observation that exogenous application of SA induces resistance in tobacco, SA has been shown to induce resistance in many plant species. Exogenous application of SA also induces expression of the same class of PR (PR-1, BGL2 [PR-2], and PR-5) genes as those induced during SAR1 A strong correlation has been observed between the in vivo increase in SA levels in infected plants and both the expression of PR genes and development of resistance. In addition SA appears to be involved in the activation of HR cell death and restriction of pathogen spread. The strongest evidence supporting the signaling role of SA in plant defense comes from studies on plants unable to accumulate SA upon pathogen infection. For example, transgenic tobacco and Arabidopsis plants constitutively expressing the Pseudomonas putida nahG gene, which encodes the SA-degrading enzyme salicylate hydroxylase, fail to develop SAR and are hypersusceptible to pathogen infection. Likewise, preventing SA accumulation by application of SA biosynthesis inhibitors also makes otherwise resistant Arabidopsis plants susceptible to Peronospora parasitica. Conversely, the elevated levels of SA present in the Arabidopsis acd (accelerated cell death; Greenberg et al. 1994; Rate et al. 1999), lsd (lesion simulating disease; Dietrich et al. 1994; Weymann et al. 1995),cpr (constitutive expressor of PR genes; Bowling et al., 1994, 1997; Clarke et al. 1998; Silva et al. 1999), ssi1 (suppressor of salicylate insensitivity of npr1-5; Shah et al. 1999), and dnd1 (defense with no HR cell death; Yu et al. 1998) mutants lead to constitutive expression of PR genes and SAR.

The Arabidopsis NPR1/NIM1 gene is an important component of the SA signal transduction pathway(s). npr1/nim1 loss-of-function mutations render the plant insensitive to. SA. SA and its functional analogs 2,6-dichloroisonicotinic acid (INA) and benzothiadiazole (BTH) are unable to induce expression of PR genes and SAR in npr1/nim1 plants. In contrast, the overexpression of NPR1 in Arabidopsis has been shown to increase resistance against bacterial and fungal pathogens. However, overexpression of NPR1 did not cause constitutive activation of defense responses. Hence, either NPR1 or possibly another coinducer may require activation by pathogen attack, or alternatively by SA before SAR can be activated NPR1 encodes a novel, 65 kD protein containing ankyrin repeats (Cao et al. 1997; Ryals et al. 1997) which are involved in its specific interaction with some members of the TGA family of bZIP DNA binding proteins.

Genetic screens for suppressors of npr1 have identified additional components of the SA signaling pathway(s). The ssi1 and sni1 mutants restore SA responsiveness and resistance in npr1 plants (Li et al. 1999; Shah et al., 1999). Elevated levels of SA are essential for the ssi1 and sni1 conferred suppression of npr1 mutant phenotypes. The SNI1 protein shows no significant homology to any known protein and has been proposed to be a negative regulator of PR gene expression and SAR. The ability of ssi1 and sni1 alleles to restore SA responsiveness in various npr1 mutant allele backgrounds argues against SSI1 and SNI1 functioning in defense as NPR1-interacting proteins. The ssi1 and sni1 mutants could suppress npr1 mutant phenotype either by somehow restoring function to the SA/NPR1 pathway or alternatively by activating an NPR1-independent, SA-response pathway.

Though NPR1 is a key component of SA signaling and overexpression of NPR1 in Arabidopsis confers enhanced resistance against pathogen, several lines of evidence suggest the existence of a NPR1-independent pathway, in addition to the NPR1-dependent pathway for SA signaling in plant defense response. For example, loss-of-function mutations in NPR1 do not confer complete loss of SA-mediated resistance. The SA-deficient NahG plants are 10-50 fold more susceptible to pathogens than the npr1 mutant Likewise, the pad4-1 mutant, which does not accumulate elevated levels of SA upon infection with virulent pathogens, is more susceptible than npr1 to powdery mildew caused by the obligate biotrophic fungus Erysiphe orontii. More recently, resistance against turnip crinkle virus in Arabidopsis has been shown to be SA dependent, but NPR1 independent (Kachroo et al. 2000). Likewise, resistance against Pseudomonas syringae in the ssi1 mutant (Shah et al. 1999) is also SA dependent, but NPR1 independent, while SA-activated resistance against P. parasitica in the constitutive SAR mutant cpr6 (Clarke et al. 1998) is due to the combined contributions of the NPR1-dependent and -independent pathways. The pathogen induced expression of PR genes in Arabidopsis is also mediated via an NPR1-independent pathway, as well as the NPR1-dependent pathway. These genes are expressed at elevated levels, albeit not at wild-type levels, in the npr1 mutant after pathogen infection. This is in contrast to the poor expression of PR genes seen in pathogen-infected NahG plants. Similarly, the pathogen induced accumulation of PAD4 transcript also occurs via SA- and NPR1-dependent, as well as SA-dependent, NPR1-independent pathways. Studies on the phytoalexin, camalexin, accumulation in Arabidopsis have also demonstrated the presence of an NPR1-independent pathway for mediating SA signaling.

Other NPR1-independent pathways also activate certain defense responses. For example, expression of the defensin gene PDF1.2 as well as resistance to the fungal pathogen Botrytis cinerea, are mediated by a pathway(s) that requires ethylene and jasmonic acid (JA), but not SA or NPR1 (Ryals et al., 1997). Additionally, an SA-dependent but NPR1-independent pathway(s) appears to regulate pathogen-induced PR gene expression in npr1 mutant plants (Yang et al., 1997; Cao et al., 1997; Shah et al., 1997) and resistance to certain pathogens (Shah et al., 1997; Glazebrook et al., 1996; Kachroo et al. 2000).

Mutant screens in Arabidopsis have made significant contributions in identifying various components of the SA signaling pathway. However, very few of these mutants have been shown to affect the NPR1-independent SA-signaling pathway. The paucity of mutants genes affecting the NPR1-independent SA signaling pathway may in part be due to the NPR1 pathway masking the role of the NPR1-independent pathway. It would be an advance in the art to develop a genetic screen capable of identifying components of the NPR1-independent SA signaling pathway. More significantly, the art would be further advanced by the identification, isolation and characterization of such components of novel defense pathways in plants.

### SUMMARY OF THE INVENTION

According to one aspect of the invention, an isolated nucleic acid molecule is provided, which comprises an SSI2 gene isolated from Arabidopsis thaliana chromosome 2 at a location within 0.2 cM from marker AthB 102 and 3.7 cM from marker GBF. The loss of function of the product of this gene is associated with altered resistance of a plant to plant pathogens or other disease-causing agents. In particular, disruption of the gene in a plant causes the plant to exhibit a phenotype comprising one or more features that include: (a) NPR1- and SA-independent constitutive expression of PR genes; (b) impairment of jasmonic acid-mediated activation of PDF1.2; and (c) accumulation of 18:0 fatty acids and decrease in 18:1 fatty acids. In various embodiments, the nucleic acid molecule may comprise a genomic clone of, or a cDNA corresponding to, the SSI2 gene of the invention. Preferably, the nucleic acid molecule encodes a polypeptide having greater than 60% (more preferably 70%, yet more preferably 80%, even more preferably 90%) identity to SEQ ID NO:3. In preferred embodiments, the nucleic acid molecule comprises a coding sequence of SEQ ID NO:1 or SEQ ID NO:2.

Also featured in the present invention is an isolated nucleic acid molecule comprising a homolog of the Arabidopsis SSI2 gene, isolated from another plant species and encoding a Δ⁹ fatty acid desaturase. In preferred embodiments the coding region of the homolog comprises a sequence which is greater than 60% (preferably 70%, yet more preferably 80%, even more preferably 90%) homologous to the coding region of SEQ ID NO:1 or SEQ ID NO:2.

According to another aspect of the invention, an isolated plant enzyme comprising a Δ⁹ fatty acid desaturase is provided. Loss of function of the enzyme in a plant results in altered resistance of the plant to plant pathogens or other disease-causing agents. Specifically, loss of function of the enzyme in a plant causes the plant to exhibit one or more features including: (a) NPR1- and SA-independent constitutive expression of PR genes; (b) impairment of jasmonic acid-mediated activation of PDF1.2; and (c) accumulation of 18:0 fatty acids and decrease in 18:1 fatty acids. In preferred embodiments, the enzyme possesses a substrate preference for 18:0 fatty acids, and its enzymatic activity produces at least one product that functions in the plant as a defense response signal molecule or a precursor of a defense response signal molecule.

Antibodies immunologically specific for the above-described plant enzyme of the invention are also provided.

The invention also features a plant-derived defense response signal molecule, produced directly or indirectly by activity of the aforementioned enzyme. This molecule, or combination of molecules, preferably comprises an 18:1 fatty acid or derivative thereof. In preferred embodiments, the defense response signal molecule inhibits a SA-independent defense response and participates in activation of a jasmonic-acid mediated defense response selected from the group consisting of activation of PDF1.2, resistance to A. brassicicola and resistance to B. cinerea.

According to another aspect of the invention, a ssi2 mutant plant is provided, which displays a phenotype characterized by one or more features including: (a) NPR1- and SA-independent constitutive expression of PR genes; (b) impairment of jasmonic acid-mediated activation of PDF1.2; and (c) accumulation of 18:0 fatty acids and decrease in 18:1 fatty acids. The phenotype is conferred by a loss-of-function mutation in the SSI2 gene.

The invention also features a method to enhance resistance of a plant to plant pathogens or other disease causing agents, comprising reducing or preventing function of a SSI2 gene product in the plant. Specifically, this method results in a plant having features that include (a) NPR1- and SA-independent constitutive expression of PR genes; (b) impairment of jasmonic acid-mediated activation of PDF1.2; and (c) accumulation of 18:0 fatty acids and decrease in 18:1 fatty acids.

Another method provided in accordance with the present invention is a method to enhance resistance of a plant to plant pathogens or other disease causing agents, comprising increasing production or activity of a SSI2 gene product in the plant.

Preferably, the enhanced resistance results from increased activity of jasmonic acid-mediated defense responses.

Fertile plants produced by any of the aforementioned methods are also provided in accordance with the invention.

Other features and advantages of the present invention will be better understood by reference to the drawings, detailed description and examples that follow.

### BRIEF DESCRIPTION OF THE DRAWINGS

FIG. 1. PR Gene Expression in ssi2 (marked in figure as ssi2-1). (FIG. 1A) Expression of the PR-1 and BGL2 genes in water-treated (W) and SA-treated (S) wild-type (SSI2 NPR1), npr1-5 (SSI2 npr1-5) and ssi2-1 (ssi2-1 NPR1 and ssi2-1 npr1-5) plants. RNA was extracted from leaves of 4 week old soil grown plants 24 h after treatment. (FIG. 1B) Comparison of constitutive PR-1, BGL2 and PR-5 expression in ssi2-1 plants homozygous for the npr1-5 (ssi2-1 npr1-5) or nim1-1 (ssi2-1 nim1-1) alleles. SSI2 plants homozygous for npr1-5 (SSI2 npr1-5) or nim1-1 (SSI2 nim1-1) served as negative controls. RNA was extracted from leaves of 4-week-old soil grown plants. Gel loading was monitored by photographing the ethidium bromide stained gel before transfer of RNA to Nytran Plus membrane. The blots were sequentially probed for the indicated genes.

FIG. 2. Growth of P. syringae in ssi2 (marked in figure as ssi2-1). P. syringae pv tomato DC3000 containing the avrRpt2 avirulence gene (OD₆₀₀ₙₘ=0.001 in 10 mM MgCl₂) was infiltrated into the abaxial surface of leaves of wild-type (SSI2 NPR1), npr1-5 (SSI2 npr1-5) and ssi2-1 (ssi2-1 NPR1 and ssi2-1 npr1-5) plants with a syringe. Four leaf discs were harvested three days later from infected leaves, weighed, ground in 10 mM MgCl₂, and bacterial numbers were titered. The bacterial numbers±SD, presented as colony-forming units (cfu) per mg leaf tissue are averages of three samples.

FIG. 3. SA and SAG Levels in ssi2 (marked in figure as ssi2-1). Leaves from 4 week-old soil-grown wild-type (SSI2 NPR1), npr1-5 (SSI2 npr1-5) and ssi2-1 (ssi2-1 NPR1 and ssi2-1 npr1-5) plants were harvested, extracted, and analyzed by HPLC. The SA and SAG values±SD, presented as micrograms of SA per gram fresh weight (FW) of tissue, are averages of three to six sets of samples per line.

FIG. 4. SA Independent Expression of ssi2-conferred Phenotypes (ssi2 marked in figure as ssi2-1). (FIG. 4A) Microscopy of trypan blue-stained leaf containing lesions from an ssi2-1 npr1-5 and ssi2-1 npr1-5 nahG plant showing intensely stained dead cells. (FIG. 4B) Comparison of constitutive PR-1 and BGL2 expression in SSI2 and ssi2-1 plants with or without the nahG transgene. All lines were either homozygous for the wild-type NPR1 or the npr1-5 mutant alleles. The wild-type (wt) or mutant (m) genotype at NPR1 and SSI2 loci is indicated on the top of the blot. Presence of the nahG transgene is indicated by the presence of a strong signal for the nahG (NahG) transcript in these lines. Gel loading was monitored by photographing the ethidium bromide stained gel before transfer of RNA to Nytran Plus membrane. The blots were sequentially probed for the indicated genes. All plants were grown in soil and sampled when 4 weeks old.

FIG. 5. Growth of P. syringae in SA-deficient SSI2 NPR1 nahG and ssi2 NPR1 nahG Plants (ssi2 marked in figure as ssi2-1). P. syringae pv tomato DC3000 containing the avrRpt2 avirulence gene (OD₆₀₀ₙₘ=0.001 in 10 mM MgCl₂) was infiltrated into the abaxial surface of leaves of wild-type (SSI2), nahG (SSI2 nahG), ssi2-1 and ssi2-1 nahG plants with a syringe. Four leaf discs were harvested three days later from infected leaves, weighed, ground in 10 mM MgCl₂, and bacterial numbers were titered. The bacterial numbers±SD, presented as colony-forming units (cfu) per mg leaf tissue are averages of three samples.

FIG. 6. Isolation of the SSI2 gene. (FIG. 6A) The locations of several recombination break points identified by CAPS analysis are designated by X. Four ORFs in the 11.7 kb region are numbered and marked by arrowheads. The number of transformants obtained with B11 and F23 clones is shown in parenthesis. (FIG. 6B) The morphological phenotype of T₂ transgenic plants complemented by the SSI2 gene in comparison with that of the ssi2 mutant (FIG. 6C) Northern blot analysis showing PR-1 gene expression in the ssi2 mutant, SSI2 (Nö) and T₁ and T₂ progeny of the F23 complemented transgenic ssi2 plant. (FIG. 6D) dCAPS analysis of same set of plants shown in FIG. 6C. (FIG. 6E) Approximately 50-60 plants of wt. ssi2 and T₂ progeny of F23 transformed ssi2 plants were spray inoculated with P. parasitica spores. Plants were sampled at 7 days post inoculation and scored as susceptible if they developed 10 or more sporangiophores per cotyledon. Cotyledons of SSI2 or ssi2/ssi2::SSI2 plants showed an average of 30-40 sporagiophores per cotyledon and 95% of these plants were susceptible. By contrast, only 5% of ssi2 plants were susceptible and they developed 2-4 fold fewer sporangiophores per cotyledon. Fungal structures and HR-like cell death were visualized by trypan blue staining. The dark-staining, round spots on SSI2 and ssi2/ssi2::SSI2 leaves are sporangiophores and the dark-staining specks on ssi2 are dead host cells.

FIG. 7. SSI2 encodes a stearoyl-ACP desaturase (S-ACP DES) with reduced activity. (FIG. 7A) A 60 aa region containing residues 121-180 was compared between S-ACP DES proteins from various plant and bacterial species. At-No is A. thaliana ecotype Nössen (also referred to herein as wild-type (wt) or SSI2) SSI2 (a portion of SEQ ID NO:3); ssi2 is polypeptide encoded by A. thaliana ssi2 mutant (SEQ ID NO:4); polypeptides from other organisms are as follows: Brassica napus (SEQ ID NO:5); Brassica juncea (SEQ ID NO:6); Ricinis (SEQ ID NO:7); Sesamum (SEQ ID NO:8); Glycine (SEQ ID NO:9); Cucumis (SEQ ID NO:10); Carthamus (SEQ ID NO:11); Arachis (SEQ ID NO:12); Solanum (SEQ ID NO:13); Oryza (SEQ ID NO:14); and Mycobacterium (SEQ ID NO:15). Variable aa are boxed and the mutated aa in ssi2 is marked by an asterisk. (FIG. 7B) Enzymatic studies were carried out with a nearly homogeneous preparation of bacterial-expressed SSI2 (Nö) and mutant proteins. Desaturase activity was determined using either 18:0 or 16:0 as a substrate. (FIG. 7C) GC-MS analysis of the double bond position in the 18:1 FA methyl ester product generated by wt (I) and mutant (II) S-ACP DES. While the scales are different for (I) and (II), the presence of 173 (X) and 217 (Y) ions are diagnostic for the two cleavage products of the derivatized 18:1^{Δ9} unsaturated FA formed by S-ACP DES.

FIG. 8. Expression of the SSI2 gene. (FIG. 8A) Histochemical staining of GUS activity in the leaves and inflorescence of transgenic plants expressing an SSI2::: GUS reporter gene. A 1631 bp fragment containing the SSI2 promoter was transcriptionally fused upstream of GUS in pBI121 and three independent transgenic lines were analyzed in both T₁ and T₂ generations. The control is a stained leaf from a wt plant. (FIG. 8B) Northern blot analysis of SSI2 (Nö), npr1-5, jar1-1 and ssi2 plants treated with water or 50 μM JA. RNA was extracted at 48 hour (h) post treatment and the blot was sequentially probed with SSI2, PDF1.2, and THI2.1. Ethidium bromide-stained rRNA served as a control for gel loading. (FIG. 8C) Northern blot analysis of plants inoculated with spores of Alternaria brassicicola. Mock (M) or fungal (A) inoculations were carried out as described previously (12). RNA was extracted at 72 h post inoculation and PDF1.2 gene expression was monitored. (FIG. 8D) Northern blot analysis of SSI2, npr1-5, jar1-1, NahG, etr1-1 and ssi2 plants treated with methanol or 50 μM MeJA. The plants were placed around a beaker containing methanol or MeJA diluted in methanol and covered with plastic wrap. RNA was prepared from leaves harvested at 48 h post treatment and analyzed for PDF1.2 gene expression.

FIG. 9. Analysis of disease resistance to B. cinerea. Infections with B. cinerea were carried out by wounding the leaves by needle pricks and subsequently spot inoculating spores at the wounded site. The number of pricks made per leaf were based on the leaf size and ranged from three per leaf for SSI2 (Nö) to one per leaf for the ssi2 mutant. Plants were treated with either water or 50 μM JA for 48 h prior to and throughout the infection and the inoculated leaves were photographed at 10 dpi.

FIG. 10. Complementation of JA-dependent PDF1.2 expression in 18:1 treated ssi2 nahG plants. Oleic acid (18:1; 0.5 mM, Sigma) or water was injected into the leaves of SSI2 (Nö), ssi2 or ssi2 nahG plants followed by treatment with 50 μM JA or water. Eight to ten individual plants each of SSI2, ssi2 or ssi2 nahG were analyzed in two independent experiments. RNA was extracted at 48 h post treatment and PDF1.2 gene expression was monitored by northern blot analysis. Ethidium bromide-stained rRNA served as a control for gel loading.

FIG. 11. Schematic diagram showing role of SSI2 in defense signaling in plants. Stearoyl-ACP desaturase encoded by SSI2 catalyzes the first step in the pathway from stearic acid (18:0) to linolenic acid (18:3), which is a precursor for signaling molecule JA. A mutation in SSI2 leads to increased levels of 18:0 and a reduction in the levels of 18:1. In addition, JA or pathogen-induced expression of defense gene PDF1.2 and resistance to B. cinerea is compromised in the ssi2 mutant. Activation of some of these JA-dependent responses may require a second signal that is generated by SSI2. Since ssi2 mutants would lack or have depressed levels of this co-activating signal, JA treatment would be insufficient to activate PDF1.2 expression or restore resistance to B. cinerea. Exogenous application of 18:1 can restore JA-inducible PDF1.2 expression, suggesting that 18:1, or an 18:1-derived signal, works in conjunction with JA to induce JA-dependent defense gene expression and pathogen resistance. The mutation in ssi2 also leads to activation of salicylic acid (SA)-mediated defense signaling. This includes constitutive PR gene expression, elevated levels of SA and resistance to both bacterial and oomycete pathogens.

### DETAILED DESCRIPTION OF THE INVENTION

I. Definitions

Various terms relating to the biological molecules and other aspects of the present invention are used throughout the specification and claims.

With respect to the genotypes of the invention, the terms “SSI2” and “ssi2” are used. The term “SSI2” is used to designate the naturally-occurring or wild-type genotype. This genotype has the phenotype of the naturally-occurring spectrum of disease resistance and susceptibility. The term “ssi2” refers to a genotype having recessive mutation(s) in the wild-type SSI2 gene. The phenotype of ssi2 individuals is enhanced resistance to selected plant pathogens by a novel defense pathway, as described in greater detail below. Where used hereinabove and throughout the specifications and claims, the term “SSI2” refers to the protein product of the SSI2 gene. The Arabidopsis SSI2 gene is exemplified herein, as is a ssi2 mutant of Arabidopsis. The mutant Arabidopsis is referred to herein either as ssi2 or ssi2-1. The wild-type, Arabidopsis is referred to herein in one of three ways: (1) as wild-type (wt), (2) as SSI2, and (3) as Nössen (Nö).

In reference to the mutant plants of the invention, the term “mutant” or “loss-of-function mutant” may be used to designate an organism or genomic DNA sequence with a mutation that causes the product of the SSI2 gene to be non-functional or largely absent. Such mutations may occur in the coding and/or regulatory regions of the SSI2 gene, and may be changes of individual residues, or insertions or deletions of regions of nucleic acids. These mutations may also occur in the coding and/or regulatory regions of other genes which may regulate or control the SSI2 gene and/or the product of the SSI2 gene so as to cause the gene product to be non-functional or largely absent. Though not exemplified herein, it should also be understood that a mutation in SSI2 can result in an increase in gene expression or in production of a protein with increased activity.

With reference to nucleic acid molecules, the term “isolated nucleic acid” may be used. This term, when applied to DNA, refers to a DNA molecule that is separated from sequences with which it is immediately contiguous (in the 5′ and 3′ directions) in the naturally occurring genome of the organism from which it was derived. For example, the “isolated nucleic acid” may comprise a DNA molecule inserted into a vector, such as a plasmid or virus vector, or integrated into the genomic DNA of a procaryote or eucaryote. An “isolated nucleic acid molecule” may also comprise a cDNA molecule.

With respect to RNA molecules, the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form (the term “substantially pure” is defined below).

With respect to proteins or peptides, the term “isolated protein (or peptide)” or “isolated and purified protein (or peptide)” may be used herein. This term refers primarily to a protein produced by expression of an isolated nucleic acid molecule of the invention. Alternatively, this term may refer to a protein which has been sufficiently separated from other proteins with which it would naturally be associated, so as to exist in “substantially pure” form.

With respect to antibodies, the term “immunologically specific” refers to antibodies that bind to one or more epitopes of a protein of interest, but which do not substantially recognize and bind other molecules in a sample containing a mixed population of antigenic biological molecules.

The term “substantially pure” refers to a preparation comprising at least 50-60% by weight the compound of interest (e.g., nucleic acid, oligonucleotide, protein, etc.). More preferably, the preparation comprises at least 75% by weight, and most preferably 90-99% by weight, the compound of interest. Purity is measured by methods appropriate for the compound of interest (e.g. chromatographic methods, agarose or polyacrylamide gel electrophoresis, HPLC analysis, and the like).

Nucleic acid sequences and amino acid sequences can be compared using computer programs that align the similar sequences of the nucleic or amino acids thus define the differences. In preferred methodologies, the BLAST programs (NCBI) and parameters used therein are employed, and the DNAstar system (Madison, Wis.) is used to align sequence fragments of genomic DNA sequences. However, equivalent alignments and similarity/identity assessments can be obtained through the use of any standard alignment software. For instance, the GCG Wisconsin Package version 9.1, available from the Genetics Computer Group in Madison, Wis., and the default parameters used (gap creation penalty=12, gap extension penalty=4) by that program may also be used to compare sequence identity and similarity.

The term “substantially the same” refers to nucleic acid or amino acid sequences having sequence variation that do not materially affect the nature of the protein (i.e. the structure, stability characteristics, substrate specificity and/or biological activity of the protein). With particular reference to nucleic acid sequences, the term “substantially the same” is intended to refer to the coding region and to conserved sequences governing expression, and refers primarily to degenerate codons encoding the same amino acid, or alternate codons encoding conservative substitute amino acids in the encoded polypeptide. With reference to amino acid sequences, the term “substantially the same” refers generally to conservative substitutions and/or variations in regions of the polypeptide not involved in determination of structure or function.

The terms “percent identical” and “percent similar” are also used herein in comparisons among amino acid and nucleic acid sequences. When referring to amino acid sequences, “percent identical” refers to the percent of the amino acids of the subject amino acid sequence that have been matched to identical amino acids in the compared amino acid sequence by a sequence analysis program. “percent similar” refers to the percent of the amino acids of the subject amino acid sequence that have been matched to identical or conserved amino acids. Conserved amino acids are those which differ in structure but are similar in physical properties such that the exchange of one for another would not appreciably change the tertiary structure of the resulting protein. Conservative substitutions are defined in Taylor (1986, J. Theor. Biol. 119:205). When referring to nucleic acid molecules, “percent identical” refers to the percent of the nucleotides of the subject nucleic acid sequence that have been matched to identical nucleotides by a sequence analysis program.

With respect to single-stranded nucleic acid molecules, the term “specifically hybridizing” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule, to the substantial exclusion of hybridization of the oligonucleotide with single-stranded nucleic acids of non-complementary sequence.

A “coding sequence” or “coding region” refers to a nucleic acid molecule having sequence information necessary to produce a gene product, when the sequence is expressed.

The term “operably linked” or “operably inserted” means that the regulatory sequences necessary for expression of the coding sequence are placed in a nucleic acid molecule in the appropriate positions relative to the coding sequence so as to enable expression of the coding sequence. This same definition is sometimes applied to the arrangement other transcription control elements (e.g. enhancers) in an expression vector.

Transcriptional and translational control sequences are DNA regulatory sequences, such as promoters, enhancers, polyadenylation signals, terminators, and the like, that provide for the expression of a coding sequence in a host cell.

The terms “promoter”, “promoter region” or “promoter sequence” refer generally to transcriptional regulatory regions of a gene, which may be found at the 5′ or 3′ side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

A “vector” is a replicon, such as plasmid, phage, cosmid, or virus to which another nucleic acid segment may be operably inserted so as to bring about the replication or expression of the segment.

The term “nucleic acid construct” or “DNA construct” is sometimes used to refer to a coding sequence or sequences operably linked to appropriate regulatory sequences and inserted into a vector for transforming a cell. This term may be used interchangeably with the term “transforming DNA”. Such a nucleic acid construct may contain a coding sequence for a gene product of interest, along with a selectable marker gene and/or a reporter gene.

The term “selectable marker gene” refers to a gene encoding a product that, when expressed, confers a selectable phenotype such as antibiotic resistance on a transformed cell.

The term “reporter gene” refers to a gene that encodes a product which is easily detectable by standard methods, either directly or indirectly.

A “heterologous” region of a nucleic acid construct is an identifiable segment (or segments) of the nucleic acid molecule within a larger molecule that is not found in association with the larger molecule in nature. Thus, when the heterologous region encodes a mammalian gene, the gene will usually be flanked by DNA that does not flank the mammalian genomic DNA in the genome of the source organism. In another example, a heterologous region is a construct where the coding sequence itself is not found in nature (e.g., a cDNA where the genomic coding sequence contains introns, or synthetic sequences having codons different than the native gene). Allelic variations or naturally-occuring mutational events do not give rise to a heterologous region of DNA as defined herein. The term “DNA construct”, as defined above, is also used to refer to a heterologous region, particularly one constructed for use in transformation of a cell.

A cell has been “transformed” or “transfected” by exogenous or heterologous DNA when such DNA has been introduced inside the cell. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

II. Description

In Arabidopsis thaliana, the NPR1 gene is required for salicylic acid (SA) induced expression of pathogenesis-related (PR) genes and systemic acquired resistance. However, loss-of-function mutations in NPR1 do not confer complete lose of SA-dependent resistance. In addition to the NPR1-dependent pathway, both resistance and PR genes expression can also be induced via an NPR1-independent pathway that heretofore had not been elucidated.

In accordance with the present invention, a novel gene, SSI2, has been identified, which is involved with a SA- and NPR1-independent pathway for expression of PR genes and resistance. The gene has been cloned and characterized. A loss-of-function mutation of SSI2 in Arabidopsis has been characterized. The features of the gene, its encoded protein and the ssi2 mutant are summarized below and are described in detail in the examples.

The recessive ssi2 mutant, identified in a genetic screen for suppressors of npr1-5, defines a new component of the NPR1-independent defense pathway. In comparison with the wild-type (SSI2 NPR1) and the npr1-5 mutant (SSI2 npr1-5) plants, the ssi2 npr1-5 double mutant and the ssi2 NPR1 single mutant constitutively express their PR (PR-1, BGL2 [PR-2], and PR-5) genes, accumulate elevated levels of SA, spontaneously develop lesions, and possess enhanced resistance to a virulent strain of P. parasitica. However, elevated levels of SA are not essential for these ssi2-conferred phenotypes, as demonstrated in the SA-deficient NahG plants. The ssi2 NPR1 nahG and ssi2 npr1-5 nahG plants retained most of the ssi2 phenotypes. In contrast to resistance against P. parasitica, while the ssi2 NPR1 plants show enhanced resistance to P. syringae as compared to the wild-type SSI2 NPR1 plants, the ssi2 npr1-5 plants were no more resistant to P. syringae, than the SSI2 npr1-5 plants. However, the ssi2 nahG plants are more resistant to P. syringae than the SSI2 nahG plants. These results suggest that SSI2 might function as a negative regulator of an SA-dependent, NPR1-independent defense pathway or alternatively an SA- and NPR1-independent defense pathway.

In Arabidopsis thaliana, the SSI2 gene is located on chromosome 2, approximately 0.2 cM from the AthB102 marker on the centromeric side and 3.7 cM from GBF on the telomeric side. Recombination analysis with these markers placed ssi2 within a 41 kb region encompassed by the bacterial artificial chromosome (BAC) F18019(Genbank Accession No. AC002333). Four open reading frames were identified within a corresponding 11.7 kb sub-region in clone F23 of a transformation competent artificial chromosome (TAC) library (Pieterse et al., 1999). Open reading frame (ORF) 2 was determined to be SSI2. Further details are set forth in Example 2.

The genomic nucleotide sequence of Arabidopsis SSI2 is set forth as SEQ ID NO: 1 or 2. The nucleotide sequence of the corresponding cDNA is set forth as SEQ ID NO:2. The predicted amino acid sequence of the encoded SSI2 protein is set forth as SEQ ID NO:3. Sequence analysis predicts that SSI2 encodes a fatty acid desaturase, of which the archetype is the stearoyl-ACP desaturase (S-ACP-DES). This enzyme is a Δ⁹ fatty acid desaturase that preferentially desaturates stearoyl-ACP (18:0 ACP). The wt SSI2 was expressed in E. coli and its gene product assayed in vitro. The encoded SSI2 enzyme had a specific activity and substrate preference (88:1 for 18- versus 16-carbon chain length FA) that are characteristic of S-ACP-DES.

The ssi2 mutant gene was analyzed by comparative sequence analysis, and was found to possess a C to T point mutation that changed the leucine (L) at position 146 of SEQ ID NO:3 to a phenylalanine (F). This mutation was found to render the enzyme 10-20-fold less active overall, but did not alter the 18- versus 16-carbon chain length substrate preference of the enzyme. The FA composition of ssi2 mutant plants reflected this change in SSI2 activity by exhibiting elevated 18:0 and decreased 18:1 FA levels, as compared with wt plants.

Further experiments with ssi2 mutant Arabidopsis plants revealed an impairment in certain of the jasmonic acid (JA)-dependent responses. Specifically, JA-mediated activation of PDF1.2, JA-mediated resistance to A. brassisicola and JA/ethylene-mediated resistance to B. cinerea were impaired in ssi2 mutants but not in wt Arabidopsis plants. In contrast, JA-mediated activation of THI2.1, a JA-inducible gene, as well as JA (or methyl JA) inhibition of root growth, were equivalent in wt and ssi2 mutant plants.

To summarize, plants carrying the recessive ssi2 mutation exhibit the following characteristics: (1) a significant decrease in activity of the encoded S-ACP-DES, resulting in elevated 18:0 FA levels at the expense of 18:1 FA; (2) constitutive activation of an NPR1-independent pathway leading to PR gene expression and resistance to P. parasitica; and (3) impairment of some JA-dependent defense responses. The fact that a defect in the FA desaturation pathway leads to activation of certain defense responses and inhibition of others indicates that one or more FA-derived signals modulates cross-talk between different defense pathways. Since the 18:1 FA pool is decreased when SSI2 activity is impaired, the FA-derived signal molecule(s) may be derived from 18:1 fatty acids. Without intending to be limited by any explanation as to mechanism, it is possible that, the FA-derived signal that co-activates certain JA-mediated defense responses also inhibits the NPR1-independent pathway. Loss of this signal in ssi2 plants would result in constitutive activation of the NPR1-independent responses. Alternatively, an increase in 18:0 content might lead to activation of lipid signaling, which could then induce the PR signal transduction pathway (23-NEED REF).

Thus, the present invention features a novel gene, SSI2, that encodes a S-ACP-DES in plants and plays a key role in modulating plant defense responses. The invention further features a FA-derived signaling molecule(s) that can be manipulated through the up- or down-regulation of the SSI2 FA desaturase, resulting in specific modifications of plant defense responses. This FA-derived signaling molecule(s) comprises at least an 18:1 FA or a derivative thereof.

Although the SSI2 genomic clone and cDNA from Arabidopsis thaliana are described and exemplified herein, this invention is intended to encompass nucleic acid sequences and proteins from other plants that are sufficiently similar to be used instead of the Arabidopsis SSI2 nucleic acid and proteins for the purposes described below. These include, but are not limited to, allelic variants and natural mutants of SEQ ID NO: 1 or 2, which are likely to be found in different species of plants or varieties of Arabidopsis. Because such variants are expected to possess certain differences in nucleotide and amino acid sequence, this invention provides an isolated SSI2 nucleic acid molecule having at least about 50% (preferably 60%, more preferably 70% and even more preferably over 80%) sequence identity in the coding regions with the nucleotide sequence set forth as SEQ ID NO: 1 or 2 (and, most preferably, specifically comprising the coding region of SEQ ID NO: 1 or 2 or the ssi2 mutant of SEQ ID NO: 1 or 2 described herein). This invention also provides isolated polypeptide products of SEQ ID NO: 1 or 2, having at least about 50% (preferably 60%, 70%, 80% or greater) sequence identity with the amino acid sequences of SEQ ID NO:3. Because of the natural sequence variation likely to exist among SSI2 genes, one skilled in the art would expect to find up to about 30-40% nucleotide sequence variation, while still maintaining the unique properties of the SSI2 gene and encoded polypeptide of the present invention. Such an expectation is due in part to the degeneracy of the genetic code, as well as to the known evolutionary success of conservative amino acid sequence variations, which do not appreciably alter the nature of the encoded protein. Accordingly, such variants are considered substantially the same as one another and are included within the scope of the present invention.

The ssi2 mutant from Arabidopsis is also part of the present invention. It exhibits an altered defense response with characteristics of regulation that have not been observed previously. This mutant is novel in its ability to constitutively express PR genes, which are known to provide defense against a wide variety of pathogens, and suppresses activation of the jasmonate-induced PDF1.2 gene.

Due to the unique phenotype conferred by the ssi2 mutation, it is easy to screen populations of mutagenized plants (e.g., by FA profile analysis) and obtain other ssi2 mutants. Such ssi2 mutants from all other species of plants are considered to be within the scope of this invention

It is contemplated that the present invention encompasses not only other plant homologs of the SSI2 gene, but also using these homologs to engineer enhanced disease resistance or to customize a defense response in other plant species. The ssi2 mutant establishes that mutations in this gene result in plants with enhanced resistance to some pathogens. Once the SSI2 homolog of a specific species is isolated, established methods exist to create transgenic plants that are deficient in the SSI2 gene product. These ssi2-like transgenic plants are also considered part of the invention.

The following sections set forth the general procedures involved in practicing the present invention. To the extent that specific materials are mentioned, it is merely for purposes of illustration and is not intended to limit the invention. Unless otherwise specified, general cloning procedures, such as those set forth in Sambrook et al., Molecular Cloning, Cold Spring Harbor Laboratory (1989) (hereinafter “Sambrook et al.”) or Ausubel et al. (eds) Current Protocols in Molecular Biology. John Wiley & Sons (2001) (hereinafter “Ausubel et al.”) are used.

III Preparation of ssi2 Mutants, SSI2 Nucleic Acids, Proteins, Antibodies and Transgenic Plants.

A. Isolation of SSI2 Genetic Mutants

Populations of plant mutants are available from which ssi2 mutants in other plant species can be isolated. Many of these populations are very likely to contain plants with mutations in the SSI2 gene. Such populations can be made by chemical mutagenesis, radiation mutagenesis, and transposon or T-DNA insertions. The methods to make mutant populations are well known in the art.

The nucleic acids of the invention can be used to isolate ssi2 mutants in other species. In species such as maize where transposon insertion lines are available, oligonucleotide primers can be designed to screen lines for insertions in the SSI2 gene. Plants with transposon or T-DNA insertions in the SSI2 gene are very likely to have lost the function of the gene product. Through breeding, a plant line may then be developed that is homozygous for the non-functional copy of the SSI2 gene. The PCR primers for this purpose are designed so that a large portion of the coding sequence the SSI2 gene are specifically amplified using the sequence of the SSI2 gene from the species to be probed (see Baumann et al., 1998, Theor. Appl. Genet. 97:729-734).

Other ssi2-like mutants can easily be isolated from mutant populations using the distinctive phenotype characterized in accordance with the present invention. This approach is particularly appropriate in, but not limited to, plants with low ploidy numbers where the phenotype of a recessive mutation is more easily detected. That the phenotype is caused by an ssi2 mutation is then established by molecular means well known in the art. Species contemplated to be screened with this approach include but are not limited to: alfalfa, aster, barley, begonia, beet, canola, cantaloupe, carrot, chrysanthemum, clover, cucumber, delphinium, grape, lawn and turf grasses, lettuce, pea, peppermint, rice, rutabaga, sorghum, sugar beet, sunflower, tobacco, tomatillo, tomato, turnip, and zinnia.

B. Isolation of SSI2 Genes

A gene can be defined by its mapped position in the plant genome. Although the chromosomal position of the gene can change dramatically, the position of the gene in relation to its neighbor genes is often highly conserved (Lagercrantz et al., 1996, Plant 3.9:13-20). This conserved micro-colinearity can be used to isolate the SSI2 gene from distantly related plant species. In accordance with the present invention, the screening of genes and markers that flank SSI2 on the chromosome are known and are farther present on the BAC and TAC clones of the Arabidopsis genome (BAC F18019, Genbank Accession No. AC002333; TAC F23, Pieterse et al., 1999). These genes and markers can be used to isolate the SSI2 gene in their midst, or to confirm the identity of an isolated SSI2 nucleic acid (described below). For example, the various coding sequences can be used to design probes to isolate the SSI2 gene on BAC clones or to map the chromosomal location of the SSI2 gene using recombination frequencies.

C. Isolation of SSI2 Nucleic Acid Molecules

Nucleic acid molecules encoding the SSI2 protein may be isolated from Arabidopsis or any other plant of interest using methods well known in the art. Nucleic acid molecules from Arabidopsis may be isolated by screening Arabidopsis cDNA or genomic libraries with oligonucleotides designed to match the Arabidopsis nucleic acid sequence of SSI2 gene (SEQ ID NO: 1 or 2). In order to isolate SSI2-encoding nucleic acids from plants other than Arabidopsis, oligonucleotides designed to match the nucleic acids encoding the Arabidopsis SSI2 protein may be likewise used with cDNA or genomic libraries from the desired species. If the SSI2 gene from a species is desired, the genomic library is screened. Alternately, if the protein coding sequence is of particular interest, the cDNA library is screened. In positions of degeneracy, where more than one nucleic acid residue could be used to encode the appropriate amino acid residue, all the appropriate nucleic acids residues may be incorporated to create a mixed oligonucleotide population, or a neutral base such as inosine may be used. The strategy of oligonucleotide design is well known in the art (see also Sambrook et al.). Alternatively, PCR (polymerase chain reaction) primers may be designed by the above method to encode a portion of the Arabidopsis SSI2 protein, and these primers used to amplify nucleic acids from isolated cDNA or genomic DNA.

In accordance with the present invention, nucleic acids having the appropriate sequence homology with an Arabidopsis SSI2 nucleic acid molecule may be identified by using hybridization and washing conditions of appropriate stringency. For example, hybridizations may be performed, according to the method of Sambrook et al. (1989, supra), using a hybridization solution comprising: 5×SSC, 5× Denhardt's reagent, 1.0% SDS, 100 μg/ml denatured, fragmented salmon sperm DNA, 0.05% sodium pyrophosphate and up to 50% formamide. Hybridization is carried out at 37-42° C. for at least six hours. Following hybridization, filters are washed as follows: (1) 5 minutes at room temperature in 2×SSC and 1% SDS; (2) 15 minutes at room temperature in 2×SSC and 0.1% SDS; (3) 30 minutes-1 hour at 37° C. in 1×SSC and 1% SDS; (4) 2 hours at 42-65° in 1×SSC and 1% SDS, changing the solution every 30 minutes.

One common formula for calculating the stringency conditions required to achieve hybridization between nucleic acid molecules of a specified sequence homology (Sambrook et al., 1989, supra) is:

Tₘ=81.5° C.+16.6 Log [Na+]+0.41(% G+C)−0.63 (% formamide)−600/#bp in duplex

As an illustration of the above formula, using [N+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C. In a preferred embodiment, the hybridization is at 37° C. and the final wash is at 42° C., in a more preferred embodiment the hybridization is at 42° C. and the final wash is at 50° C., and in a most preferred embodiment the hybridization is at 42° C. and final wash is at 65° C., with the above hybridization and wash solutions. Conditions of high stringency include hybridization at 42° C. in the above hybridization solution and a final wash at 65° C. in 0.1×SSC and 0.1% SDS for 10 minutes.

Nucleic acids of the present invention may be maintained as DNA in any convenient cloning vector. In a preferred embodiment, clones are maintained in plasmid cloning/expression vector, such as pBluescript (Stratagene, La Jolla, Calif.), which is propagated in a suitable E. coli host cell.

Arabidopsis SSI2 nucleic acid molecules of the invention include DNA, RNA, and fragments thereof which may be single- or double-stranded. Thus, this invention provides oligonucleotides (sense or antisense strands of DNA or RNA) having sequences capable of hybridizing with at least one sequence of a nucleic acid molecule encoding the protein of the present invention. Such oligonucleotides are useful as probes for detecting Arabidopsis SSI2 genes or transcripts.

D. Engineering Plants to Alter SSI2 Activity

Though the ssi2 mutant Arabidopsis exemplified herein is an EMS-induced mutant, any plant may be transgenically engineered to display a similar phenotype. While the natural ssi2 mutant has lost the functional product of the SSI2 gene due to a single point mutation, a transgenic plant can be made that also has a similar loss of the SSI2 product. This approach is particularly appropriate to plants with high ploidy numbers, including but not limited to wheat, corn and cotton.

A synthetic mutant can be created by a expressing a mutant form of the SSI2 protein to create a “dominant negative effect”. While not limiting the invention to any one mechanism, this mutant SSI2 protein will compete with wild-type SSI2 protein for interacting proteins in a transgenic plant. By over-producing the mutant form of the protein, the signaling pathway used by the wild-type SSI2 protein can be effectively blocked. Examples of this type of “dominant negative” effect are well known for both insect and vertebrate systems (Radke et al, 1997, Genetics 145:163-171; Kolch et al., 1991, Nature 349:426-428).

A second kind of synthetic mutant can be created by inhibiting the translation of the SSI2 mRNA by “post-transcriptional gene silencing”. The SSI2 gene from the species targeted for down-regulation, or a fragment thereof, may be utilized to control the production of the encoded protein. Full-length antisense molecules can be used for this purpose. Alternatively, antisense oligonucleotides targeted to specific regions of the SSI2-encoded RNA that are critical for translation may be utilized. The use of antisense molecules to decrease expression levels of a pre-determined gene is known in the art. Antisense molecules may be provided in situ by transforming plant cells with a DNA construct which, upon transcription, produces the antisense RNA sequences. Such constructs can be designed to produce full-length or partial antisense sequences. This gene silencing effect can be enhanced by transgenically over-producing both sense and antisense RNA of the gene coding sequence so that a high amount of dsRNA is produced (for example see Waterhouse et al., 1998, PNAS 95:13959-13964). In a preferred embodiment, part or all of the SSI2 coding sequence antisense strand is expressed by a transgene. In a particularly preferred embodiment, hybridizing sense and antisense strands of part or all of the SSI2 coding sequence are transgenically expressed.

A third type of synthetic mutant can also be created by the technique of “co-suppression”. Plant cells are transformed with a copy of the endogenous gene targeted for repression. In many cases, this results in the complete repression of the native gene as well as the trausgene. In a preferred embodiment, the SSI2 gene from the plant species of interest is isolated and used to transform cells of that same species.

Transgenic plants displaying enhanced SSI2 activity can also be created. This is accomplished by transforming plant cells with a transgene that expresses part of all of an SSI2 coding sequence, or a sequence that encodes the either the SSI2 protein or a protein functionally similar to it. In a preferred embodiment, the complete SSI2 coding sequence is transgenically over-expressed.

Transgenic plants with one of the transgenes mentioned above can be generated using standard plant transformation methods known to those skilled in the art. These include, but are not limited to, Agrobacterium vectors, polyethylene glycol treatment of protoplasts, biolistic DNA delivery, UV laser microbeam, gemini virus vectors, calcium phosphate treatment of protoplasts, electroporation of isolated protoplasts, agitation of cell suspensions in solution with microbeads coated with the transforming DNA, agitation of cell suspension in solution with silicon fibers coated with transforming DNA, direct DNA uptake, liposome-mediated DNA uptake, and the like. Such methods have been published in the art. See, e.g., Methods for Plant Molecular Biology (Weissbach & Weissbach, eds., 1988); Methods in Plant Molecular Biology (Schuler & Zielinski, eds., 1989); Plant Molecular Biology Manual (Gelvin, Schilperoort, Verma, eds., 1993); and Methods in Plant Molecular Biology—A Laboratory Manual (Maliga, Klessig, Cashmore, Gruissem & Varner, eds., 1994).

The method of transformation depends upon the plant to be transformed. Agrobacterium vectors are often used to transform dicot species. Agrobacterium binary vectors include, but are not limited to, BIN19 (Bevan, 1984) and derivatives thereof, the pBI vector series (Jefferson et al., 1987), and binary vectors pGA482 and pGA492 (An, 1986) For transformation of monocot species, biolistc bombardment with particles coated with transforming DNA and silicon fibers coated with transforming DNA are often useful for nuclear transformation.

DNA constructs for transforming a selected plant comprise a coding sequence of interest operably linked to appropriate 5′ (e.g., promoters and translational regulatory sequences) and 3′ regulatory sequences (e.g., terminators). In a preferred embodiment, the coding region is placed under a powerful constitutive promoter, such as the Cauliflower Mosaic Virus (CaMV) 35S promoter or the figwort mosaic virus 35S promoter. Other constitutive promoters contemplated for use in the present invention include, but are not limited to: T-DNA mannopine synthetase, nopaline synthase (NOS) and octopine synthase (OCS) promoters.

Transgenic plants expressing a sense or antisense SSI2 coding sequence under an inducible promoter are also contemplated to be within the scope of the present invention. Inducible plant promoters include the tetracycline repressor/operator controlled promoter, the heat shock gene promoters, stress (e.g., wounding)-induced promoters, defense responsive gene promoters (e.g. phenylalanine ammonia lyase genes), wound induced gene promoters (e.g. hydroxyproline rich cell wall protein genes), chemically-inducible gene promoters (e.g., nitrate reductase genes, glucanase genes, chitinase genes, etc.) and dark-inducible gene promoters (e.g., asparagine synthetase gene) to name a few.

Tissue specific and development-specific promoters are also contemplated for use in the present invention. Examples of these included, but are not limited to: the ribulose bisphosphate carboxylase (RuBisCo) small subunit gene promoters or chlorophyll a/b binding protein (CAB) gene promoters for expression in photosynthetic tissue; the various seed storage protein gene promoters for expression in seeds; and the root-specific glutamine synthetase gene promoters where expression in roots is desired.

The coding region is also operably linked to an appropriate 3′ regulatory sequence. In a preferred embodiment, the nopaline synthetase polyadenylation region (NOS) is used. Other useful 3′ regulatory regions include, but are not limited to the octopine (OCS) polyadenylation region.

Using an Agrobacterium binary vector system for transformation, the selected coding region, under control of a constitutive or inducible promoter as described above, is linked to a nuclear drug resistance marker, such as kanamycin resistance. Other useful selectable marker systems include, but are not limited to: other genes that confer antibiotic resistances (e.g., resistance to hygromycin or bialaphos) or herbicide resistance (e.g., resistance to sulfonylurea, phosphinothricin, or glyphosate).

Plants are transformed and thereafter screened for one or more properties, including the lack of SSI2 protein, SSI2 mRNA, constitutive HR-like lesions or expression of PR genes, altered FA metabolism or enhanced resistance to a selected plant pathogen, such as P. parasitica. It should be recognized that the amount of expression, as well as the tissue-specific pattern of expression of the transgenes in transformed plants can vary depending on the position of their insertion into the nuclear genome. Such positional effects are well known in the art. For this reason, several nuclear transformants should be regenerated and tested for expression of the transgene.

Transgenic plants that exhibit one or more of the aforementioned desirable phenotypes can be used for plant breeding, or directly in agricultural or horticultural applications. Plants containing one transgene may also be crossed with plants containing a complementary transgene in order to produce plants with enhanced or combined phenotypes.

E. In Vivo Synthesis of the SSI2 Protein

The availability of amino acid sequence information, such as the full length sequence in SEQ ID NO: 2, enables the preparation of a synthetic gene that can be used to synthesize the Arabidopsis SSI2 protein in standard in vivo expression systems, or to transform different plant species. The sequence encoding Arabidopsis SSI2 from isolated native nucleic acid molecules can be utilized. Alternately, an isolated nucleic acid that encodes the amino acid sequences of the invention can be prepared by oligonucleotide synthesis. Codon usage tables can be used to design a synthetic sequence that encodes the protein of the invention. In a preferred embodiment, the codon usage table has been derived from the organism in which the synthetic nucleic acid will be expressed. For example, the codon usage for pea (Pisum sativum) would be used to design an expression DNA construct to produce the Arabidopsis SSI2 in pea. Synthetic nucleic acid molecules may be prepared by the phosphoramadite method employed in the Applied Biosystems 38A DNA Synthesizer or similar devices, and thereafter may be cloned and amplified in an appropriate vector.

The availability of nucleic acids molecules encoding the Arabidopsis SSI2 enables production of the protein using in vivo expression methods known in the art. According to a preferred embodiment, the protein may be produced by expression in a suitable expression system. The SSI2 protein of the present invention may also be prepared by in vitro transcription and translation of either native or synthetic nucleic acid sequences that encode the proteins of the present invention. While in vitro transcription and translation is not the method of choice for preparing large quantities of the protein, it is ideal for preparing small amounts of native or mutant proteins for research purposes, particularly since in vitro methods allow the incorporation of radioactive nucleotides such as ³⁵S-labeled methionine. The SSI2 proteins of the present invention may be prepared by various synthetic methods of peptide synthesis via condensation of one or more amino acid residues, in accordance with conventional peptide synthesis methods. The SSI2 produced by native cells or by gene expression in a recombinant procaryotic or eukaryotic system may be purified according to methods known in the art.

F. Antibodies Immunospecific for SSI2

The present invention also provides antibodies that are immunologically specific to the Arabidopsis SSI2 of the invention. Polyclonal antibodies may be prepared according to standard methods. In a preferred embodiment, monoclonal antibodies are prepared, which are specific to various epitopes of the protein. Monoclonal antibodies may be prepared according to general methods of Köhler and Milstein, following standard protocols. Polyclonal or monoclonal antibodies that are immunologically specific for the Arabidopsis SSI2 can be utilized for identifying and purifying SSI2 from Arabidopsis and other species. For example, antibodies may be utilized for affinity separation of proteins for which they are specific or to quantify the protein. Antibodies may also be used to immunoprecipitate proteins from a sample containing a mixture of proteins and other biological molecules.

IV. Use of SSI2 Nucleic Acids, SSI2 Proteins and Antibodies, ssi2 Mutants and Transgenic Plants.

A. Uses of SSI2 Nucleic Acids

SSI2 nucleic acids may be used for a variety of purposes in accordance with the present invention. DNA, RNA, or fragments thereof may be used as probes to detect the presence and/or expression of SSI2 genes. Methods in which SSI2 nucleic acids may be utilized as probes for such assays include, but are not limited to: (1) in situ hybridization; (2) Southern hybridization (3) Northern hybridization; and (4) assorted amplification reactions such as polymerase chain reactions (PCR).

The SSI2 nucleic acids of the invention may also be utilized as probes to identify related genes from other plant species. As is well known in the art, hybridization stringencies may be adjusted to allow hybridization of nucleic acid probes with complementary sequences of varying degrees of homology. As described above, SSI2 nucleic acids may be used to advantage to produce large quantities of substantially pure SSI2, or selected portions thereof.

The SSI2 nucleic acids can be used to identify and isolate further members of this novel disease resistance signal transduction pathway in vivo. A yeast two hybrid system can be used to identity proteins that physically interact with the SSI2 protein, as well as isolate their nucleic acids. In this system, the sequence encoding the protein of interest is operably linked to the sequence encoding half of a activator protein. This construct is used to transform a yeast cell library which has been transformed with DNA constructs that contain the coding sequence for the other half of the activator protein operably linked to a random coding sequence from the organism of interest. When the protein made by the random coding sequence from the library interacts with the protein of interest, the two halves of the activator protein are physically associated and form a functional unit that activates the reporter gene. In accordance with the present invention, all or part of the Arabidopsis SSI2 coding sequence may be operably linked to the coding sequence of the first half of the activator, and the library of random coding sequences may be constructed with cDNA from Arabidopsis and operably linked to the coding sequence of the second half of the activator protein. Several activator protein/reporter genes are customarily used in the yeast two hybrid system. In a preferred embodiment, the bacterial repressor LexA DNA-binding domain and the Gal4 transcription activation domain fusion proteins associate to activate the LacZ reporter gene (see Clark et al., 1998, PNAS 95:5401-5406). Kits for the two hybrid system are also commercially available from Clontech, Palo Alto Calif., among others.

B. Uses of SSI2 Proteins and Antibodies

The SSI2 proteins of the present invention can be used to identify molecules with binding affinity for SSI2, which are likely to be novel participants in this resistance pathway. In these assays, the known protein is allowed to form a physical interaction with the unknown binding molecule(s), often in a heterogenous solution of proteins. The known protein in complex with associated molecules is then isolated, and the nature of the associated protein(s) and/or other molecules is determined.

Antibodies that are immunologically specific for SSI2 may be utilized in affinity chromatography to isolate the SSI2 protein, to quantify the SSI2 protein utilizing techniques such as western blotting and ELISA, or to immuno-precipitate SSI2 from a sample containing a mixture of proteins and other biological materials. The immuno-precipitation of SSI2 is particularly advantageous when utilized to isolate affinity binding complexes of SSI2, as described above.

C. Uses of ssi2 Mutants

The ssi2 mutants of the invention display a unique combination of defense responses that include constitutive HR and expression of PR genes and enhanced disease resistance to certain plant pathogens, and therefore can be used to improve crop and horticultural plant species by customizing the defense response. Plants species contemplated in regard to this invention include, but are not limited to: alfalfa, aster, barley, begonia, beet, canola, cantaloupe, carrot, chrysanthemum, clover, corn, cotton, cucumber, delphinium, grape, lawn and turf grasses, lettuce, pea, peppermint, rice, rutabaga, sorghum, sugar beet, sunflower, tobacco, tomatillo, tomato, turnip, wheat, and zinnia.

The ssi2 mutant of Arabidopsis exhibits constitutive activation of an NPR1-independent pathway leading to PR gene expression, and a constitutive HR. It is therefore contemplated that the ssi2 mutants will exhibit broad-spectrum resistance against a wide range of fungal, bacterial and viral pathogens. Such pathogens include, but are not limited to: P. syrinzgae and P. parasitica.

The ssi2 mutants of the invention can be used to identify and isolate additional members of this disease resistance pathway. Mutations that, when combined with ssi2, suppress the ssi2 phenotype, are likely to interact directly with SSI2, or to compensate in some other way for the loss of SSI2 function.

D. Uses of SSI2 Transgenic Plants

The transgenic plants of the invention are particularly useful in conferring the SSI2 phenotype to many different plant species. In this manner, a host of plant species with enhanced or modified defense responses can be made, to be used as breeding lines or directly in commercial operations. Such plants can have uses as crop species, or for ornamental use.

A plant that has had functional SSI2 transgenically depleted should exhibit a defense response profile similar to that of the ssi2 Arabidopsis mutant described above. A transgenic approach is advantageous because it allows ssi2-phenotype plants to be created quickly, without time-consuming mutant generation, selection, and back-crossing. Transgenically created ssi2-phenotype plants have special utility in polyploid plants, such as wheat, where recessive mutations are difficult to detect.

A plant with increased functional SSI2, produced either by over-expression of an endogenous gene, expression of a transgene or other means of modifying the activity of SSI2, are expected to have additional defense response properties consistent with increased production of the SSI2-associated FA-derived signal molecule(s) discovered in accordance with the present invention. It is already known that transgenic plants expressing a yeast Δ⁹ FA desaturase exhibit increased resistance to a wide variety of plant pathogens, including fungi such as Erisyphe, Phytophthora, Verticillium and Fusarium, bacteria such as Pseudomonas, and viruses such as tobacco mosaic virus (U.S. Pat. No. 6,225,528 to Chin et al., the entirety of which is incorporated by reference herein). Similarly, plants that have increased production or activity of the SSI2 FA desaturase may be expected to display broad resistance to various plant pathogens. Further, since the inventors have discovered that the JA pathway is modulated by the FA-derived signal associated with SSI2 activity, it can be predicted that plants overproducing SSI2 will display enhanced JA-mediated defense responses. In addition, one of skill in the art would expect the SSI2 gene, which is a plant gene, to be more effectively expressed in plants than would be genes from other species. Accordingly, transgenic plants transformed with SSI2 are likely to be superior in defense responses that the yeast Δ⁹ desaturase-transgenic plants disclosed in U.S. Pat. No. 6,225,528.

The following examples are provided to illustrate embodiments of the invention. They are not intended to limit the scope of the invention in any way.

### EXAMPLE 1

### Loss-of-Function Mutation in the Arabidopsis SSI2 Gene Confers SA- and NPR1-Independent Expression of PR Genes and Resistance Against Bacterial and Oomycete Pathogens.

Genetic screens for SAR mutants in the npr1 background were used as a means to identify genes functioning in an NPR1-independent pathway. In this example, a mutant that identifies a new component of an NPR1-independent defense pathway is characterized.

Methods

Growth conditions for plant and bacteria. Arabidopsis plants were grown in soil at 22° C. in growth chambers programmed for a 16-h light (8000 to 10,000 lux) and 8-h dark cycle unless otherwise stated. P. syringae pv. tomato DC3000 carrying a plasmid-borne avrRpt2 gene was propagated at 30° C. on King's B medium containing rifampicin (100 mg/ml) and kanamycin (25 mg/ml). P. parasitica isolate Emco5 was cultivated on the susceptible ecotype Nössen.

Bacterial infection of plants. Plants for infection with P. syringae pv. tomato DC3000 were grown in soil at 22° C. in a growth chamber programmed for 12-h light and 12-h dark cycle. Three days before infection the plants were transferred to a 16-h light and 8-h dark cycle infection with P. syringae pv. tomato DC3000 carrying a plasmid-bome avrRpt2 gene were performed as described earlier (Shah et al. 1997). Four leaves per plant were infiltrated with a suspension (OD₆₀₀ of 0.001) in 10 mM MgCl₂. 12 leaf discs, 0.5 cm in diameter (0.20 cm²) were harvested at the indicated time and placed in pre-weighed tubes. After the weight of each sample was determined the samples were processed for bacterial counts and RNA extraction as described earlier (Shah et al. 1997). Bacterial counts were expressed at colony forming units per mg leaf tissue.

Inoculation with the virulent P. parasitica isolate Emco5 was done on seven-day-old soil grown plants, using spore suspensions of P. parasitica. Plants were sprayed with a freshly prepared suspension of conidiospores in water (10⁶ spores/ml). Inoculated plants were kept covered with a clear plastic dome to maintains high humidity throughout the course of the experiment, and fungal growth was evaluated under a dissecting microscope eight days post inoculation by counting the number of sporangiophores per leaf. Plants with leaves containing 5 or more sporangiophores were scored as infected.

Chemical treatment of plants. Four-week-old plants were sprayed and sub-irrigated with a solution of SA (500 mM) or BTH (100 mM active ingredient) in water as previously described (Shah et al. 1997). As controls, plants were similarly treated with water. Leaves were harvested at the indicated times after treatment and quick-frozen in liquid nitrogen. Leaf samples were stored at −80° C.

RNA extraction, northern and dot blot analyses. Large-scale preparation of RNA from Arabidopsis was carried out Small-scale extraction of RNA from one or two leaves was performed in the TRIzol reagent (GIBCO-BRL, Gaithersburg, Md.) following manufacturer's instructions. Northern blot analysis and synthesis of random primed probes for PR-i, BGL2 and PR-5 were synthesized as described earlier (Shah et al. 1997).

Histochemistry and microscopy. Leaf samples for trypan blue staining and epifluorescence microscopy were obtained from three-week-old soil grown plants. Trypan blue staining on P. parasitica infected leaves was carried out on samples harvested eight days post inoculation. Samples were processed and analyzed.

SA and SAG estimations. SA and SAG were extracted and estimated from 0.25 to 0.5 g of fresh weight leaf tissue.

Mutagenesis and selection of ssi2 mutant. M₂ seeds derived from ethyl methyl sulfonate mutagenized npr1-5 seeds (ecotype Nö) were screened for constitutive PR gene expression as previously described (Shah et al. 1999).

Genetic analysis. Backcrosses were performed by pollinating flowers of npr1-5 (SSI2 npr1-5) plant with pollen from a ssi2 npr1-5 double mutant plant. For all other genetic analyses progeny from a backcrossed line homozygous for the ssi2 and npr1-5 mutant alleles was used. To generate ssi2 plants homozygous for the NPR1 wild-type allele, pollen from a ssi2 npr1-5 double mutant were used to pollinate flowers from an Arabidopsi ecotype Nö line 1/8E/5 (Shah et al., 1997) which is wild, type at both the SSI2 and NPR1 loci. Likewise, to generate ssi2 plants homozygous for the nim1-1 mutant allele, pollen from a ssi2 npr1-5 double mutant were used to pollinate flowers from a SSI2 nim1-1 plant (ecotype Wassilewskija). Success of the cross was confirmed by CAPS analysis on F₁ plants for heterozygosity at the APR1 locus. Segregation of the ssi2 mutant allele was monitored in the F₂ progeny by the presence of the small lesion plus plant phenotype and by northern blot or dot blot analysis for constitutive PR-1 gene expression. CAPS analysis was performed as previously described (Shah et al., 1999) on DNA from these phenotypically ssi2 plants to identify plants homozygous for the wild-type NPR1 or the nim1-1 mutant allele. For mapping analysis, pollen from a ssi2 npr1-5 double mutant (ecotype Nö) was used to pollinate flowers from a wild-type plant of the ecotype Columbia F₂ progeny plants from the above cross were monitored for spontaneous lesion and constitutive PR-1 expression phenotype by dot blot analysis. DNA for PCR was isolated from leaf tissue and used for CAPS or SSLP marker analysis. ssi2 mutant lines containing the nahG transgene were generated by fertilizing flowers from a ssi2 npr1-5 plant with pollen from a transgenic NahG plant (ecotype Nö). Success of the cross was confirmed by analyzing expression of the nahG gene in the F₁ plants. A quarter of the F₂ plants had the ssi2 conferred lesion⁺ phenotype suggesting that the nahG gene did not suppress the lesion⁺ phenotype of ssi2 plants. Northern blot analysis showed constitutive expression of PR genes in these plants, reaffirming that these were truly ssi2 plants. Northern blot analysis also identified expression of the nahG gene in roughly three quarter of these plants. Analysis of the F₃ progeny of some of these F₂ lines identified F₂ plants that were homozygous for ssi2, NPR1 and nahG.

Results

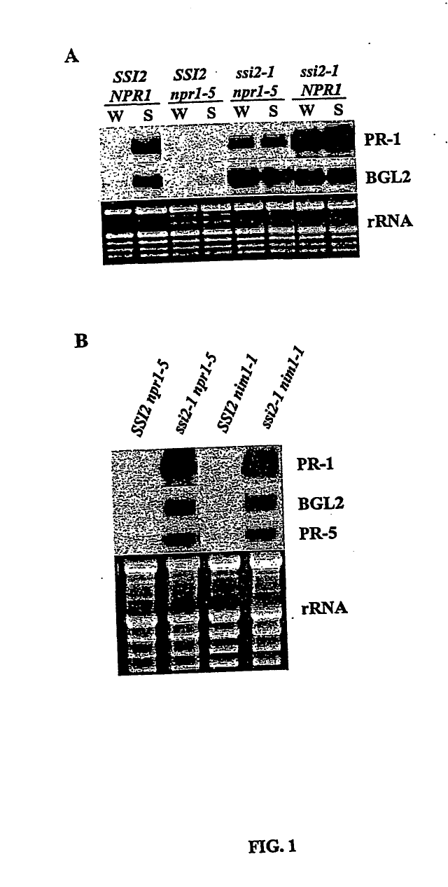

Loss-of-function mutations in the SSI2 gene confer constitutive PR gene expression and spontaneous development of HR-like lesions in npr1-5 plants. The ssi2 mutant was isolated in a screen for suppressors of the SA-insensitive npr1-5 mutant; the details of this screen have been previously described (Shah et al., 1999). Briefly, three-to four-week old M₂ progeny of an ethyl methyl sulfonate mutagenized population of npr1-5 were screened by RNA blot analysis for mutants that constitutively accumulated elevated levels of the PR (PR-1, BGL2, and PR-5) gene transcripts. As shown in FIGS. 1A and 1B, unlike the wild-type (SSI2 NPR1) and the npr1-5 mutant (SSI2 npr1-5), the ssi2 npr1-5 double mutant constitutively expressed the PR-1, BGL2 and PR-5 gene transcripts at elevated levels. Exogenous application of SA did not further increase accumulation of the PR gene transcripts in the ssi2 npr1-5 plant. As compared to npr1-5 and wild-type plants, the ssi2 npr1-5 double mutant plants were smaller, had curled leaves with more prominent indentations of the leaf margin, and spontaneously developed necrotic lesions (data not shown). These lesions are associated with the increased accumulation of autofluorescent material and dead cells, suggesting that these are HR-like lesions. The ssi2 phenotypes were weaker when plants were grown under short (8 h light cycle) versus long (16 h light cycle) day photoperiod cycle (data not shown).

Genetic characterization of ssi2. F₁ plants derived from a back cross of the ssi2 npr1-5 double mutant to the SSI2 npr1-5 parent lacked all ssi2-conferred phenotypes suggesting ssi2 was recessive to the wild-type SSI2 allele. Analysis of F₂ progeny confirmed that the ssi2-conferred phenotypes are due to a recessive mutation in a single gene; the ssi2 phenotype segregated in a 3 PR-1⁻ lesion⁻:1 PR-1⁺ lesion⁺ Mendelian ratio (81 PR-1⁻ lesion⁻ plants to 26 PR-1⁺ lesion⁺ plants; c²=0.02; 0.9>P>0.5).

A second site mutation within the npr1-5 allele could potentially suppress the npr1-5 loss-of-function phenotype. However, the recessive nature of ssi2 argues against ssi2 plants containing an intragenic mutation within the npr1-5 allele. This was further confirmed by demonstrating that ssi2 segregates independent of the npr1-5 allele. In the F₂ progeny of a cross between ssi2 npr1-5 and SSI2 NPR1, ssi2 NPR1 segregants were recovered. The presence of NPR1 allele in the F₂ plants was analyzed by CAPS as previously described (Shah et al., 1999). Furthermore, while NPR1 maps on chromosome 1, SSI2 maps on chromosome 4 (0.5 cM from AthB102 and 5.2 cM from GBF) further confirming that ssi2 is not an intragenic suppressor of npr1-5. In comparison with ssi2 npr1-5 plants, constitutive expression of the PR-1 gene was repeatedly observed to be higher in ssi2 NPR1 plants (FIG. 1A). Unlike PR-1, BGL2 (FIG. 1A) and PR-5 (data not shown) were generally expressed at comparable levels in the ssi2 npr1-5 and ssi2 NPR1 plants. However, in a few experiments BGL2 and PR-5 expression were observed to be higher in the ssi2 NPR1 plants as compared to ssi2 npr1-5 plants.

To test whether the ssi2-conferred phenotypes require NPR1 protein we analyzed ssi2-conferred phenotypes in nim1-1 plants (allelic with npr1). A single base pair insertion in nim1-1 is expected to cause premature termination of NPR1, resulting in a truncated protein lacking the C-terminal 349 amino acids. ssi2 nim1-1 plants expressed the PR genes at levels comparable to those observed in ssi2 npr1-5 plants (FIG. 1B). Furthermore, like the ssi2 npr1-5 plant, the ssi2 nim1-1 plant also developed HR-like lesions (data not shown). These results strongly argue that the ssi2 mutant phenotypes do not require the NPR1 protein.

ssi2 confers enhanced resistance to P. parasitica and P. syringae.

PR gene expression is an useful marker for resistance response. Since the ssi2 mutant constitutively expresses PR genes we tested whether it also shows enhanced resistance against pathogens. The ssi2 mutant is in the Arabidopsis ecotype Nössen. The fungal pathogen isolate Emco5 is virulent on Arabidopsis ecotype Nö, causing extensive growth and sporulation. As shown in Table 1, while wild-type Arabidopsis ecotype Landsberg plants, used as resistant controls, did not show any fungal growth, both wild-type Nö (SSI2 NPR1) and npr1-5 (SSI2 npr1-5, Nö background) plants are highly susceptible, supporting profuse fungal growth and sporulation. In contrast, the majority of the ssi2 NPR1 (150 out of 160) and ssi2 npr1-5 plants (160 out of 173) did not support fungal growth (Table 1).

TABLE 1
Disease ratings of SSI2 and ssi2 plants after inoculation with
P. parasitica biotype Emco5.

| | Total number | | | Average number |
|---|---|---|---|---|
| | of plants | | | of sporangiophores/ |
| Genotype^{a} | inoculated | Diseased^{b} | Healthy | cotyledon |
| SSI2 NPR1 | 120 | 113 | 7 | 30-40 |
| (Nö) | | | | |
| SSI2 NPR1 | 200 | 0 | 200 | 0 |
| (Ler) | | | | |
| SSI2 npr1-5 | 146 | 142 | 4 | 30-40 |
| ssi2 NPR1 | 160 | 10 | 150 | 10-20 |
| ssi2 npr1-5 | 173 | 13 | 160 | 10-20 |
| SSI2 NPR1 | 109 | 109 | 0 | >50 |
| nahG | | | | |
| ssi2 NPR1 | 145 | 70 | 75 | 10-20 |
| nahG | | | | |

^{a}All plants were homozygous for the desginated alleles.
^{b}Plants were scored as diseased if the inoculated leaves showed presence of 5 or more sporangiophores per cotyledon.

In the few cotyledons of ssi2 NPR1 and ssi2 npr1-5 plants where growth and sporulation were observed, the number of sporangiophores per cotyledon were 2-3 fold slower than that observed in cotyledons of infected SSI2 NPR1 and SSI2 npr1-5 plants.

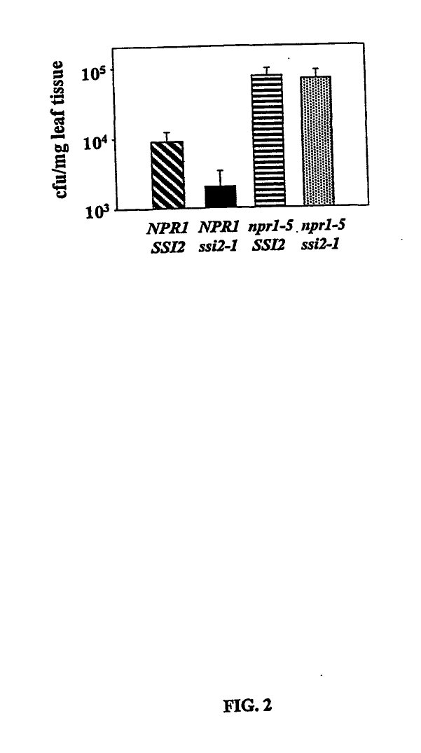

It has been shown that the npr1-5 mutant shows enhanced susceptibility to the bacterial pathogen P. syringae pv tomato carrying the avirulence gene avrRpt2 (Shah et al. 1997, 1999). Furthermore, the ssi1 mutant, which restored SA responsiveness in npr1-5 plants, also restored resistance against avirulent P. syringae pv tomato in npr1-5 plants suggesting that the mutation in ssi1 somehow activates signaling downstream of NPR1 in the SA signaling pathway. Similarly, the mutation in ssi2 may likewise activate signaling downstream of NPR1 and restore resistance against P. syringae pv tomato. We therefore compared the growth of P. syringae pv tomato carrying the AvrRpt2 avirulence gene in ssi2 npr1-5, SSI2 npr1-5 and ssi2 NPR1 plants, and as control in wild-type SSI2 NPR1 plant. As shown in FIG. 2, while the mutation in ssi2 enhanced resistance approximately five-fold in plants containing the wild-type NPR1 allele, it did not enhance resistance in the plants containing the npr1-5 allele. This result suggests that, unlike ssi1, NPR1 is required for the ssi2-conferred enhanced resistance against P. syringae pv tomato.

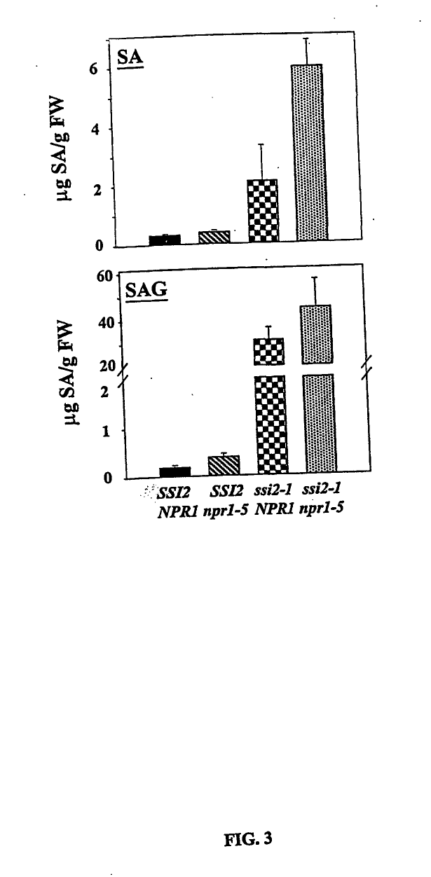

The ssi2 mutant constitutively accumulates high levels of SA and SAG. Several previously described mutants constitutively expressing PR genes and resistance constitutively accumulate elevated levels of SA. The high levels of endogenous SA are required for the phenotypes of these mutants. For example, the cpr6 mutant accumulates elevated levels of SA, expresses PR genes and confers enhanced resistance against P. syringae and P. parasitica. Like ssi2, while PR gene expression in cpr6 is NPR1 independent, resistance to P. syringae requires NPR1. The cpr6 phenotypes are dependent on its ability to accumulate elevated levels of SA. It is likely that, like cpr6, the enhanced resistance against P. syringae and the constitutive PR expression observed in ssi2 plants could be due to the ssi2 mutant accumulating elevated levels of SA. We therefore tested the levels of SA and its glucoside (SAG) in SSI2 and ssi2 plants. As shown in FIG. 3, in plants homozygous for the ssi2 mutant allele, ssi2 NPR1 and ssi2 npr1-5, SA levels were 7- and 15-fold higher than in the SSI2 NPR1 and SSI2 npr1-5 plants, respectively. Likewise, SAG levels were >100-fold higher in the ssi2 plants as compared to SSI2 plants.

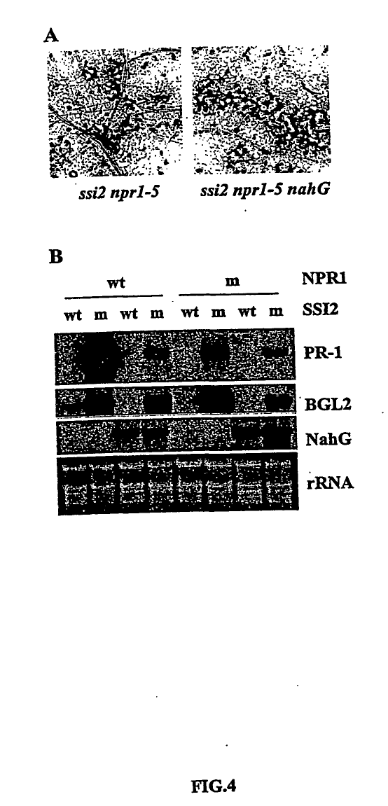

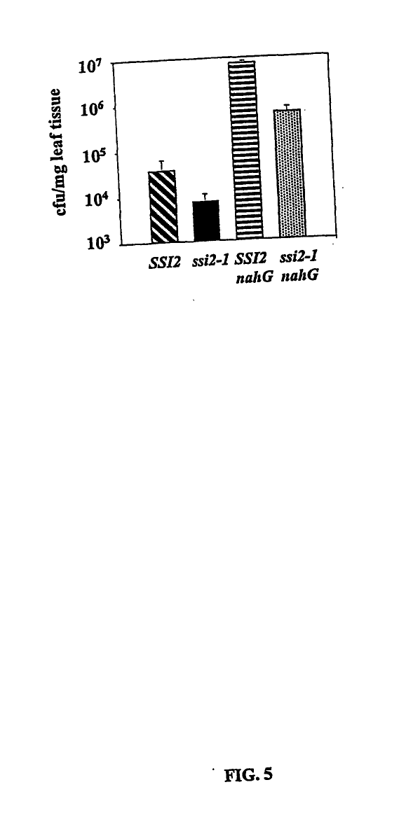

SA is not essential for the ssi2-conferred phenotypes. To determine if the elevated levels of endogenous SA are required for the ssi2-conferred phenotypes we crossed a ssi2 plant with a transgenic plant (NahG) expressing the SA-degrading enzyme salicylate hydroxylase. Previously we have shown that this particular NahG line prevents high level accumulation of SA and SAG in the ssi1 mutant and suppress ssi1-conferred phenotypes. F₂ progeny of a cross between ssi2 and a SSI2 nahG showed a 3:1 segregation of lesion⁻: lesion⁺ plants. Furthermore, the ssi2 small plant phenotype and constitutive PR expression cosegregated with the lesion− phenotype. Approximately three quarters of these ssi2-like plants also expressed the nahG transcript suggesting high levels of SA and SAG are not required for the ssi2-conferred phenotypes. These results were confirmed in the F₃ generation. The ssi2-conferred spontaneous lesions were present in plants homozygous for ssi2 and the nahG transgene (FIG. 4A). In addition, PR genes were also constitutively expressed at elevated levels in ssi2 NPR1 nahG and ssi2 npr1-5 nahG plants (FIG. 4B). In contrast, these genes were not expressed at elevated levels in SSI2 NPR1, SSI2 npr1-5, SSI2 NPR1 nahG and SSI2 npr1-5 nahG plants. However, the presence of the nahG transgene did lower the absolute levels of accumulation of the PR-1 transcripts in ssi2 NPR1 nahG and ssi2 npr1-5 nahG plants as compared to ssi2 NPR1 and ssi2 npr1-5 plants, respectively, suggesting SA enhanced the ssi2-conferred constitutive PR-1 expression. BTH treatment of the ssi2 NPR1 nahG plants increased the accumulation of PR-1 transcript to a level similar to those seen in the untreated ssi2 NPR1, and BTH-treated wild-type (SSI2 NPR1) plants (data not shown), confirming the role of SA in enhancing the ssi2-conferred constitutive PR-1 phenotype. The effect of nahG on ssi2-conferred BGL2 and PR-5 expression was more variable, with nahG having no effect on the levels of BGL2 and PR-5 expression in two out of four experiments. SA also enhances the small plant size phenotype of ssi2. The ssi2 NPR1 nahG and ssi2 npr1-5 nahG plants were slightly larger and developed visible lesions later than ssi2 plants lacking the nahG transgene (data not shown).

ssi2 confers resistance to P. parasitica and P. syringae in the SA-deficient NahG plants. Since elevated levels of SA and SAG are not essential for the manifestation of ssi2-conferred constitutive PR gene expression and lesion phenotypes even though they can affect these phenotypes, we tested whether ssi2-conferred resistance was also independent of SA accumulation. Resistance against P. parasitica Emco5 and P. syringae tomato carrying the avirulence gene AvrRpt2 were compared among SSI2 NPR1, SSI2 NPR1 nahG, ssi2 NPR1 and ssi2 NPR1 nahG plants. As shown in Table 1, SSI2 NPR1 nahG plants are hypersusceptible to P. parasitica Emco5 as compared to the susceptible SSI2 NPR1 plant of ecotype Nössen. Not only did all the SSI2 NPR1 nahG plants show disease symptoms but they also supported higher levels of sporulation. Furthermore, the newly emerging leaves also showed the presence of sporangiophores (data not shown). Interestingly, the ssi2 allele partially restored resistance in the ssi2 NPR1 nahG plants. Approximately 50% of ssi2 NPR1 nahG plants showed little or no signs of infection. Furthermore, the ssi2 NPR1 nahG plants that did show infection had 2-3 fold fewer sporangiophores compared to the SSI2 NPR1 nahG plants.

The SSI2 NPR1 nahG plants were also hypersusceptible to P. syringae pv tomato carring the avirulence gene avrRpt2, suppporting ˜250 fold more bacterial growth than the SSI2 NPR1 plants. However, presence of the ssi2 allele in the ssi2 NPR1 nahG plants partially restored resistance (20-fold increase).

Discussion

To identify components of the SA signal transduction pathway we had earlier successfully set up a screen in Arabidopsis thaliana for suppressors of npr1 (Shah et al. 1999). The ssi1 mutation, previously identified in this screen, restored SA-responsive PR gene expression and resistance in npr1 plants. In this example we describe the characterization of the ssi2 mutant, identified in the same screen, which uncovers an NPR1-independent pathway for expression of PR genes and resistance. Loss-of-function mutations in SSI2 confer constitutive PR gene expression and enhanced resistance against the oomycete fungus P. parasitica Emco5 and the bacterial pathogen P. syringae pv tomato. In addition, ssi2 plants are smaller than the wild-type SSI2 plants, and spontaneously develop HR-like lesions. SA is not required for expression of ssi2 phenotypes.

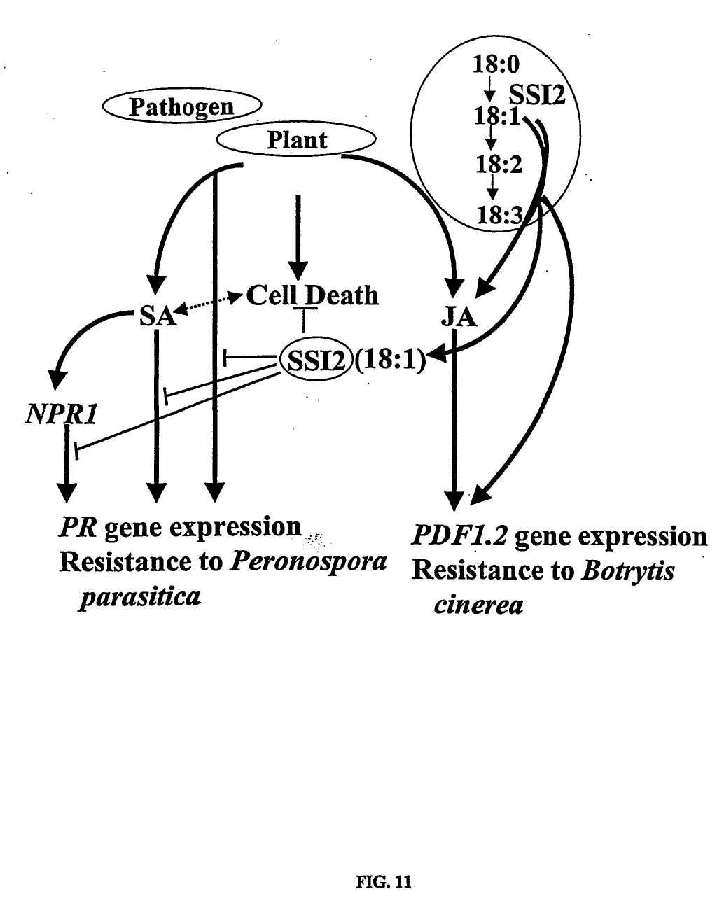

Constitutive PR expression in ssi2 is due to activation of NPR1-dependent and NPR1-independent defense pathways. NPR1 is not required for the ssi2-conferred constitutive expression of PR genes, which are expressed at elevated levels in ssi2 npr1-5 and ssi2 nim1-1 plants (FIGS. 1A and 1B). However, in comparison to ssi2 npr1-5, presence of NPR1 enhanced accumulation of the PR-1 transcipt in ssi2 NPR1 plants (FIG. 1A). This increased expression of PR-1 in ssi2 NPR1 plants is likely due to elevated levels of SA (FIG. 3) activating signaling through the NPR1 pathway. This is supported by the observation that salicylate hydroxylase expression repeatedly reduced PR-i expression in ssi2 NPR1 nahG plants (FIG. 4B). Furthermore, BTH application increased PR-1 expression in ssi2 NPR1 nahG plants but not in ssi2 npr1-5 nahG plants (data not shown). These results suggest ssi2 activates SA signaling through the NPR1-dependent as well as NPR1-independent pathways (FIG. 11). The existence of an NPR1-independent SA signaling pathway has previously been reported. For example, while npr1 mutants do not express PR genes at elevated levels in response to exogenously applied SA or its analogs, PR genes are expressed at elevated levels in pathogen-infected npr1 plants. This is in contrast to the poor expression of PR genes seen in pathogen-infected NahG plants. Similarly, the pathogen activated accumulation of PAD4 transcript also occurs via a SA-dependent, NPR1-independent pathway besides the SA- and NPR1-dependent pathway.

While the npr1 mutation reduced ssi2-conferred constitutive PR-1 expression, it however had little if any effect on constitutive BGL2 and PR-5 expression (FIGS. 1A and 4B, and data not shown). Thus, while the NPR1-dependent pathway is the primary activator(s) of PR-1 expression in ssi2 plants, BGL2 and PR-5 expression are induced primarily by the NPR1-independent pathway. Similar differences in the requirement of NPR1 for PR-1 expression, as compared to BGL2 and PR-5 expression, have previously been noted in pathogen infected npr1 plants.

ssi2-conferred constitutive PR expression is not dependent on SA accumulation. Like ssi2, constitutive expression of PR genes in the ssi1, sni1, cpr6 and acd6 mutants also occurs independently of NPR1. However, SA is required for constitutive PR gene expression in these mutants. This is in contrast to the SA-independent expression of PR genes in the ssi2 NPR1 nahG and ssi2 npr1-5 nahG plants (FIG. 4B). SA and SAG levels in the ssi2 NPR1 nahG and ssi2 npr1-5 nahG plants were comparable to those seen in uninfected wild-type plants (data not shown). Moreover, the NahG transgenic line used in these experiments is hypersusceptible to P. syringae and P. parasitica (FIG. 5; Table 1) and has previously been shown by us to suppress the constitutive PR gene expression phenotype of the ssi1 mutant (Shah et al. 1999). Since the ssi1 and ssi2 mutants accumulate comparable levels of SA and SAG it is highly unlikely that the residual SA and SAG in ssi2 NPR1 nahG and ssi2 npr1-5 nahG plants activate PR gene expression. Thus SSI2 negatively regulates the NPR1-independent pathway at a step downstream of SA (FIG. 11). Alternatively, the ssi2 phenotypes might be due to activation of an SA- and NPR1-independent pathway.

ssi2 confers enhanced resistance against P. parasitica and P. syringae in the absence of SA accumulation. In Arabidopsis resistance against the oomycete fungus P. parasitica and the bacterial pathogen P. syringae requires SA. Loss of SA accumulation due to expression of the SA degrading salicylate hydroxylase causes hypersusceptibility to these pathogens in transgenic NahG plants (FIG. 5). The mutation in ssi2 confers enhanced resistance against these pathogens. Interestingly, while the ssi2-conferred resistance against P. parasitica Emco5 was independent of NPR1 (Table 1), enhanced resistance against an avirulent strain of P. syringae pv tomato was dependent on NPR1. These seemingly contradictory results can be explained if resistance against P. syringae is primarily dependent on the NPR1-dependent SA signal transduction pathway, while resistance against P. parasitica is conferred primarily by the NPR1-independent SA signal transduction pathway. Since SSI2 acts as a negative regulator of the NPR1-independent pathway at a step downstream of SA action (FIG. 11), the lack of SSI2 repressor activity in ssi2 plants will allow significant level of signaling through this pathway even in the absence of elevated SA levels. This could account for the strong resistance against P. parasitica observed in ssi2 npr1-5 plants. Constitutive signaling through this NPR1-independent pathway could also account for the partial resistance against P. syringae, observed in npr1-5 plants in comparison with NahG plants (FIGS. 3 and 5). Furthermore, presence of NPR1 plus elevated levels of SA will additionally activate signaling through the NPR1-dependent resistance pathway leading to further increase in resistance against P. syringae in ssi2 APR1 plants as compared to ssi2 npr1-5 plants. Resistance towards P. syringae and P. parasitica in cpr6 has been shown similarly to be differentially regulated by NPR1-dependent plus NPR1-independent pathways (Clarke et al. 1998). However, unlike cpr6, significant resistance against P. syringae and P. parasitica is observed in ssi2 nahG plants as compared to SSI2 nahG plants (FIG. 5 and Table 1) further supporting the argument that SSI2 represses signaling through the NPR1-independent pathway at a step after SA action. This model of SSI2 action also explains why resistance in the ssi2 NPRL nahG plants is not as strong as in ssi2 NPRL plants; the NPR1-dependent defense pathway cannot be activated in the ssi2 NPR1 nahG plant as SA does not accumulate to high levels. Furthermore, since ssi2 is not a null allele but retains residual SSI2 repressor activity, this could account for the difference in resistance between ssi2 NPR1 and ssi2 NPR1 nahG plants.

An alternative explanation for the enhanced resistance against P. syringae and P. parasitica in ssi2 NPR1 nahG and ssi2 npr1-5 nahG plants is offered by SSI2 functioning in a SA-independent pathway. While the SA dependent pathway is the primary pathway governing resistance against these pathogens activation of this alternative SA-independent pathway might confer some resistance against these pathogens in the absence of SA accumulation. Recently, a SA independent pathway, requiring ethylene and JA signaling has been proposed to induce systemic resistance (ISR) in response to colonization of Arabidopsis roots with Pseudomonas fluorescens Pieterese et al. 1996, 1998).

SA is not required for the development of HR-like lesions in ssi2.

ssi2 plants spontaneously develop HR-like lesions. Spontaneous lesions have been observed in several Arabidopsis mutants exhibiting constitutive SAR In all these cases lesion formation was associated with elevated levels of SA and SAG. In case of the ssi1 (Shah et al. 1999), acd6 (Rate et al. 1999), cep (Silva et al. 1999), lsd1 (Dangl et al. 1996), lsd6 and lsd7 (Weymann et al 1995) mutants spontaneous lesion formation has been shown to require elevated SA levels. However, lesion formation is independent of SA in the lsd2, lsd4 (Hunt et al. 1997) and cpr5 (Bowling et al. 1997) mutants. Similar to lsd2, lsd4 and cpr5 plants, lesion formation in ssi2 plants is not dependent on elevated levels of SA (FIG. 5A). Lesion formation in ssi2 could be a result of the metabolic stress caused by constitutively active defense responses. Spontaneous lesions have been observed in plants exposed to metabolic stress. For example, expression of the bacterial proton pump bacterio-opsin, a subunit of the cholera toxin gene and yeast vacuolar invertase, and inhibition of protoporphyrinogen oxidase in plants causes the development of lesions. Furthermore, in these cases lesion formation is associated with the accumulation of elevated levels of SA and SAG. Spontaneously occurring cell death in turn might cause increased SA accumulation in these plants. This is in agreement with the existence of a feedback amplification loop involving cell death and SA in plants. Alternatively, both SA accumulation and cell death may occur independently of each other as a direct response to the metabolic stress in ssi2 plants.

In summary, we have shown that suppressor screens are useful in identifying not only additional components of a given pathway but also components of parallel pathways. Through the study of a loss-of-function ssi2 mutant we have identified a novel gene, which functions to repress signaling downstream of SA in an NPR1-independent defense pathway. ssi2-conferred constitutive PR gene expression, resistance and spontaneous cell death do not require SA, however, SA accentuates these ssi2 phenotypes. We have also shown that the contribution of the NPR1-dependent and NPR1-independent pathways towards resistance depends on the pathogen. The ssi2 mutant to our knowledge is the only mutant known to activate resistance against P. syringae and P. parasitica in NahG plants. Resistance to these pathogens has previously been shown to be SA dependent.

### EXAMPLE 2

SSI2 Encodes a Fatty Acid Desaturase that Modulates the Activation of Defense Signaling Pathways in Plants.

To identify components of the NPR1-independent signaling pathway, we performed a genetic screen for suppressors of the npr1-5 mutation. Through this process, the recessive ssi2 mutation was identified and shown to confer constitutive expression of PR-1, PR-2 and PR-5, spontaneous lesion formation, constitutive SA accumulation, enhanced resistance to P. parasitica and P. syringae pv tomato and a stunted growth morphology (Example 1). This example describes experiments designed to elucidate the function of the SSI2 protein

Methods

Genetic analysis. A ssi2/ssi2 plant derived from Arabidopsis ecotype Nössen was crossed to a SSI2/SSI2 (wt) plant from the Columbia (Col-0) ecotype. CAPS (Konieczny & Ausubel 1993) and SSLP (Bell & Ecker, 1994) marker analyses were performed on 656 F₂ progeny that, based on their morphology and PR-1 gene expression, were homozygous for the ssi2 mutation This analysis placed ssi2 on chromosome 2, approximately 0.2 cM from AthB102 on the centromeric side and 3.7 cM from GBF on telomeric side. Using sequence information generated by the Arabidopsis genome project, 14 additional CAPS markers spanning this region were generated and used to further delimit the region containing ssi2.

dCAPS analysis. A 100 bp fragment was amplified using PCR primers p1 (5′-AGAGAGGGCTAGAGAGCTCCCTG-3′; SEQ ID NO:16) and p2 (5′-AGTGTTCAACATAGTTTGATAGGTCCTAA-3′; SEQ ID NO:17) from the chromosomal DNA of wt, mutant, and T₁ or T₂ progenies of ssi2/ssi2::SSI2 transgenic plants. The bases underlined in p2 were present as “GG” in the original sequence; this modification created a Dde I site in the PCR product amplified from wt DNA. Since the ssi2 mutation alters the 3′ base flanking AA of p2, no Dde I site is present in the PCR product amplified from ssi2 DNA.

RNA extraction and northern analyses. Small-scale extraction of RNA from one or two leaves was performed in the TRIzol reagent (GIBCO-BRL, Gaithersburg, Md.) following the manufacturer's instructions. Northern blot analysis and synthesis of random primed probes for PR-1, BGL2 and PR-5, PDF1.2 and THI2.1 were synthesized.

Arabidopsis transformation. TAC, BAC, pBI121, pBin19 (Xiang et al., 1999) or pVK18 (Moore et al., 1998) derived clones were moved into Agrobacterium tumefaciens strains GV3101 or MP90 by electroporation and were used to transform Arabidopsis via the floral dip method (Clough & Bent, 1998). Selection of transformants was carried out on media containing hygromycin or kanamycin.

Expression in Escherichia coil, in vitro S-ACP desaturase assay, and GC-MS analysis. The putative signal peptide region of SSI2 was predicted by aligning it with the protein sequence from castor bean S-ACP DES. cDNA's from both wt and ssi2 were amplified such that they lacked N-terminal 34 aa of the putative signal peptide and the 35th aa was converted to a methionine. The cDNA's were isolated as a NcoI/EcoRI-linkered PCR products and cloned into pET-28a vector. Purification and determination of desaturase activity were performed. Dimethyl disulfide adducts of fatty acid methyl esters were prepared. Methyl esters of unsaturated FA and their dimethyl disulfide derivatives were identified by MS analysis.

Results

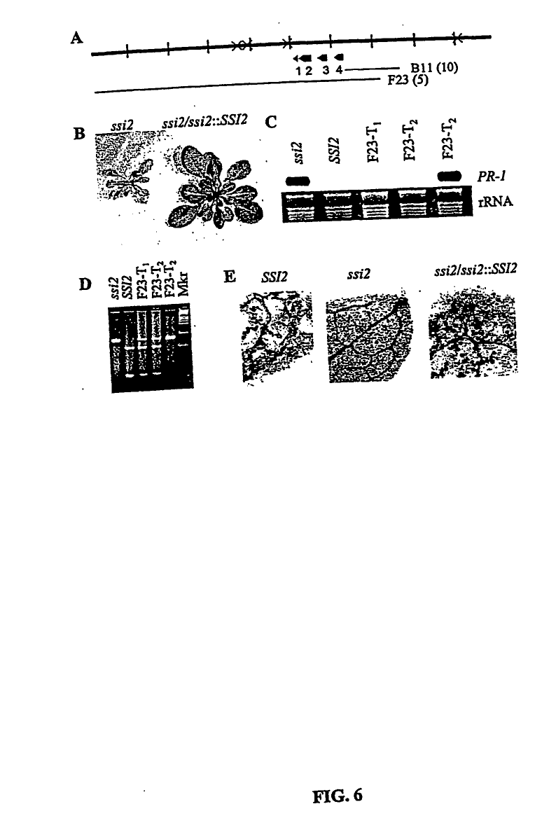

Positional cloning of SSI2. Through co-dominant cleaved amplified polymorphic sequence (CAPS) and simple sequence length polymorphic (SSLP) marker analysis, the ssi2 gene was mapped to a 41 kb region of chromosome 2 that is encompassed by the bacterial artificial chromosome (BAC) clone F18019 (FIG. 6A). To identify the SSI2 gene, ssi2 npr1-5 double mutant plants were transformed with subclones of F18O19 that had been inserted into a binary-BAC (BIBAC) vector (Hamilton, 1997). Alternatively, these plants were transformed with overlapping clones from a transformation-competent artificial chromosome (TAC) library (Liu et al., 1999) that hybridized to a 2 kb polymerase chain reaction (PCR)-generated probe corresponding to open reading frame (ORF) 4 within the 41 kb region. Transformants were screened for restoration of the wt morphology and absence of constitutive PR-1 gene expression. Only TAC clone F23 complemented the ssi2 mutation (FIGS. 6B and 6C). Furthermore, in 105 T₂ progeny from 5 independently derived F23-transformed T₁ lines, the presence or absence of the hygromycin selectable marker correlated with the development of the SSI2 or the ssi2 phenotype, respectively (FIG. 6C).

Based on the complementation and recombination analyses, the SSI2-containing region of F23 was reduced to 11.7 kb. This region contains 4 ORFs, which were amplified by PCR and sequenced. Comparison with sequences from wt Nö plants revealed only one difference; a C to T transition was detected in ORF2. Since this variation between wt and ssi2 sequences could not be distinguished by restriction enzyme polymorphism, a derived-CAPS (dCAPS) marker (Neff et al., 1998) was used to confirm the identity of the ssi2 mutation. Analysis of 63 T₂ progeny from the ssi2/ssi2::SSI2 complementing lines showed that stunted growth and constitutive PR-1 gene expression co-segregated with the ssi2-specific band pattern (FIGS. 6C, 6D). The presence of the SSI2 gene also correlated with a loss of ssi2-induced resistance to P. parasitica Emco5; those plants containing the hygromycin marker gene were as susceptible as the wt controls, while those lacking the marker were resistant (FIG. 6E). Final confirmation that the SSI2 gene was isolated came from the demonstration that both a genomic clone and a CaMV ³⁵S promoter-driven cDNA clone of ORF2 restored wt morphology to ssi2 plants (data not shown).

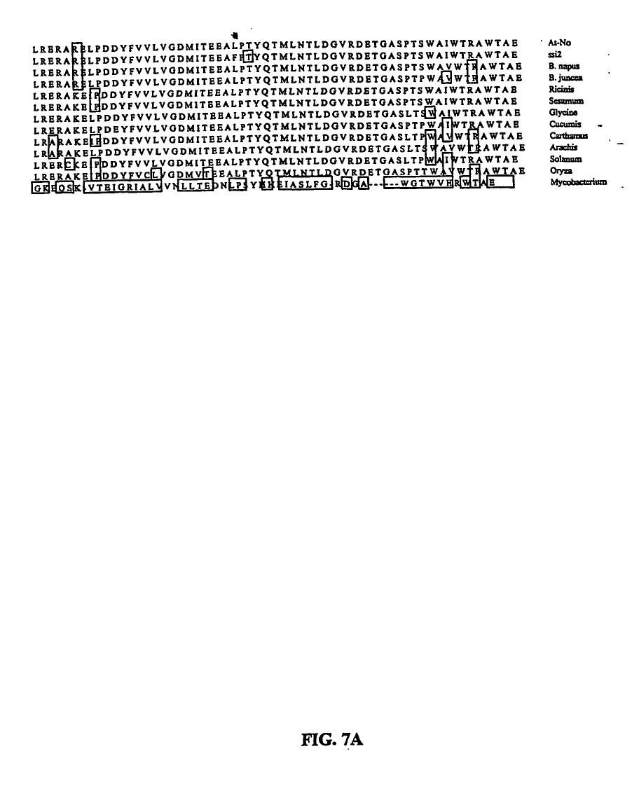

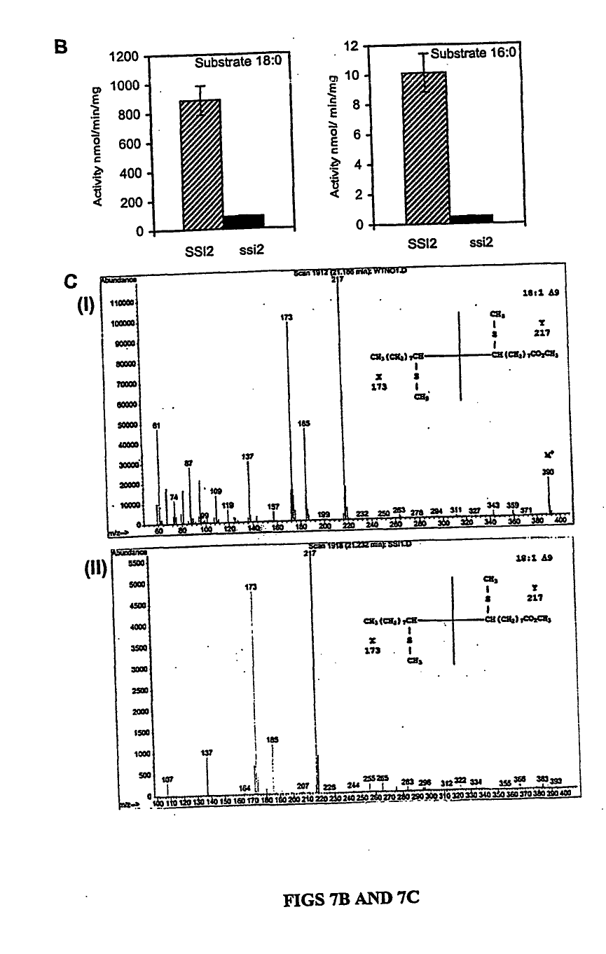

The ssi2 mutant exhibits reduced S-ACP DES activity. Sequence analysis predicted that SSI2 encodes a member of the soluble fatty acid (FA) desaturase enzyme family (FIG. 7A) (Penninckx et al., 1996). These enzymes are key regulators of FA biosynthesis, of which the archetype is the S-ACP-DES. S-ACP-DES preferentially desaturates stearoyl-ACP (18:0-ACP) between carbons 9 and 10, yielding oleoyl-ACP (18:1^{Δ9}-ACP). To define the functional identity of the SSI2 gene product, the wt gene was expressed in Escherichia coli and the activity of the purified enzyme was assessed by in vitro assays. Wt SSI2 had specific activity (˜800 nm/min/mg; FIG. 7B) and substrate preference (88:1, for 18 versus 16 carbon chain length FA) characteristic of S-ACP-DES. Gas chromatography-mass spectroscopy (GC-MS) analysis confirmed the regiospecificity as Δ⁹ (FIG. 7C).

The C to T mutation in ssi2 changes the leucine (L) at amino acid (aa) position 146 to a phenylalanine (F). Comparison of 24 S-ACP-DES proteins from various plants revealed that all, except ssi2, contain a leucine at this position (data not shown). The high degree of conservation for L₁₄₆, combined with the recessive nature of the ssi2 mutation, suggested that ssi2 might have reduced and/or altered enzymatic activity. In contrast to the wt, the mutant protein was approximately 10- and 20-fold less active on both 18:0 and 16:0 substrates, respectively, but the 18:16 substrate preference ratio and the Δ⁹ regiospecificity were unaltered (FIG. 7).

The FA composition in ssi2 plants is altered. To determine whether reduced S-ACP DES activity affects the FA composition in ssi2 plants, the levels of various 16 and 18 carbon fatty acids were monitored using GC-MS (Table 2). Leaves of the ssi2 mutant contained considerably elevated levels of 18:0 compared to the wt and decreased levels of 16:3, 18:1 and 18:2 (Table 2). The levels of other FAs including 18:3 were similar to or slightly reduced from those observed in wt plants. The presence of nearly wt levels of these FAs in ssi2 plants is likely due to the activity of other S-ACP DES isoforms, several of which have been identified in Arabidopsis (Maleck & Dietrich, 1999).

TABLE 2
Fatty acid composition of total leaf lipids from wild type and ssi2.
All measurements were made on 22° C. grown plants and data
are described as mol % ± standard error calculated for a
sample size of six.

| Fatty Acid | Wild Type | ssi2 |
|---|---|---|
| 16:0 | 19.9 ± 1.0 | 18.1 ± 0.7 |
| 16:1-trans | 2.7 ± 0.1 | 2.2 ± 0.3 |
| 16:2 | 0.3 ± 0.0 | 0.2 ± 0.0 |
| 16:3 | 9.9 ± 0.7 | 6.3 ± 0.2 |
| 18:0 | 1.1 ± 0.1 | 13.4 ± 1.7 |
| 18:1 | 2.7 ± 0.1 | 0.9 ± 0.2 |
| 18:2 | 18.1 ± 0.4 | 14.9 ± 0.6 |
| 18:3 | 44.8 ± 1.0 | 43.5 ± 2.0 |

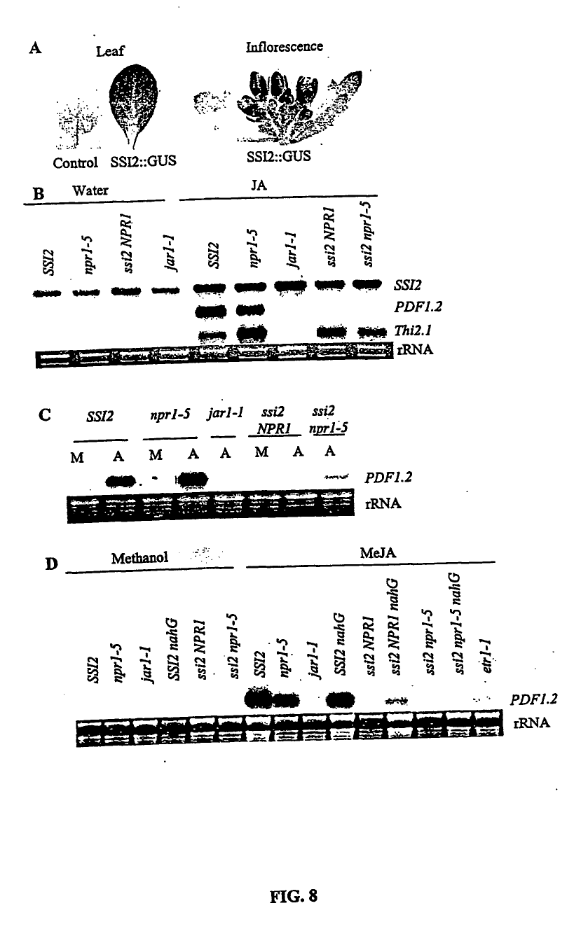

Activation of some JA-inducible defense responses is impaired in ssi2 plants. S-ACP-DES catalyzes the first step in the pathway from stearic acid (18:0) to linolenic acid (18:3), and linolenic acid is a precursor for the defense signaling molecule JA (Farmer & Ryan, 1992). Since JA is required to activate the wounding response and defenses against insect pests and certain microbial pathogens, we monitored SSI2 gene expression after wounding, pathogen infection or treatment with SA, JA or ethylene. Analysis of transgenic plants expressing β-glucuronidase (GUS) driven by the SSI2 promoter revealed that this promoter is active in all tissues studied, with the highest level of expression detected in flowers (FIG. 8A). Northern analysis further indicated that SSI2 gene expression was not affected by the ssi2 or npr1-5 mutations, or in the presence of the NahG transgene, which encodes salicylate hydroxylase (FIG. 8B and data not shown). It also did not increase substantially over basal levels at 12, 24 or 48 hours after treating plants with SA, JA, ethylene, wounding or infection with turnip crinkle virus (data not shown).

The ability of ssi2 plants to activate various JA-dependent defense responses was then assessed. Although JA-treatment activated PDF1.2 expression effectively in wt and npr1-5 plants, it induced only low to undetectable levels of PDF1.2 expression in ssi2 NPR1, ssi2 npr1-5, or JA-insensitive jar1-1 mutant plants (FIG. 8B). By contrast, IA-induced activation of the THI2.1 gene and inhibition of root growth by JA or its derivative methyl JA (MeJA), was unaffected in ssi2 plants (FIG. 8B and data not shown). Inoculation with A. brassicicola induced strong expression of PDF1.2 in wt and npr1-5 plants, but only low to no expression in ssi2 plants (FIG. 8C). Since loss of PDF1.2 inducibility could be due to antagonism by the elevated SA levels found in ssi2 mutants, we analyzed PDF1.2 expression in ssi2 nahG plants. The presence of the NahG transgene did not restore wt levels of PDF1.2 expression in MeJA-treated (FIG. 8D) or A. brassicicola-inoculated ssi2 NPR1 or ssi2 npr1-5 plants (data not shown). Thus, the reduction of PDF1.2 inducibility in ssi2 nahG plants is not due to elevated SA levels. Since PDF1.2 expression is dependent on concomitant activation of the ethylene and JA signaling pathways, we also tested whether ethylene signaling is altered in the ssi2 mutant. A treatment of 10 or 20 parts per million of ethylene induced PDF1.2 expression in wt plants, but not in ssi2 plants (data not shown). However, ssi2 plants were highly susceptible to infection by A. brassicicola, which is pathogenic on JA-insensitive but not ethylene-insensitive mutants. Based on this result, the mutation in S-ACP DES does not appear to perturb the ethylene signaling pathway.

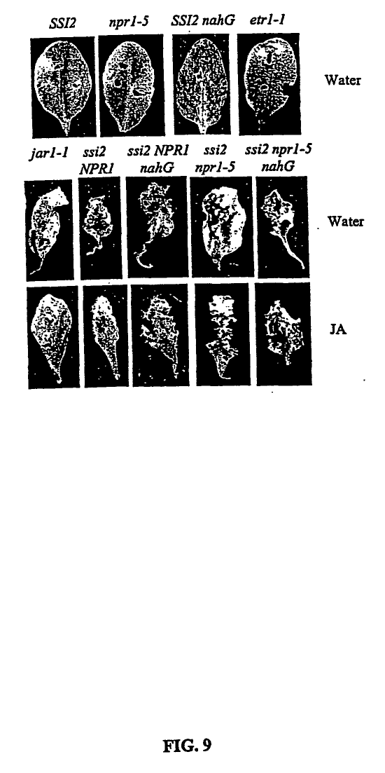

In addition to PDF1.2 expression, resistance to B. cinerea, which is mediated by JA- and ethylene-dependent pathways, was impaired in ssi2 NPR1 and ssi2 npr1-5 plants (FIG. 9). Exogenously applied JA or MeJA failed to restore B. cinerea resistance on ssi2 or ssi2 nahG plants. Indeed, the symptoms exhibited by these plants were as severe as those displayed by jar1-1 mutants. By contrast, ethylene-insensitive etr1-1 plants displayed moderate symptoms and wt, npr1-5, and NahG transgenic plants were fully resistant.

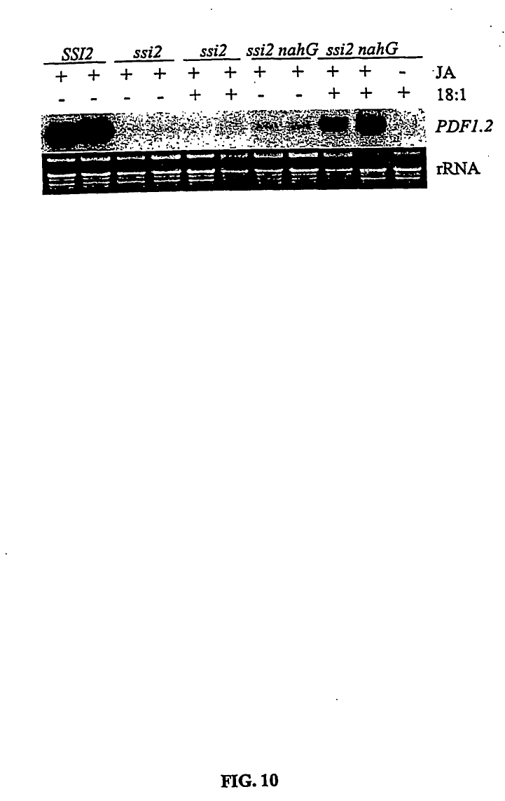

JA plus 18:1 induce PDF1.2 expression in ssi2 nahG plants. A likely explanation for the failure of JA to activate PDF1.2 and resistance to B. cinerea in ssi2 nahG plants is that certain JA-dependent responses require a second signal that is generated by S-ACP DES. ssi2 or ssi2 nahG plants would lack or have reduced levels of this co-activating signal. Consistent with this hypothesis, treatment of ssi2 nahG plants with a combination of JA and 18:1 activated PDF1.2 (FIG. 10). ssi2 plants failed to respond to JA plus 18:1 (which is reduced three fold in ssi2), probably because of antagonistic effects of the high levels of endogenous SA.

Discussion

The recessive ssi2 mutation was identified as a suppressor of the npr1-5 allele. In this example, we describe the cloning and characterization of the SSI2 gene. Based on sequence analysis and biochemical assays, we demonstrate that SSI2 encodes S-ACP DES. This enzyme, along with other soluble FA desaturases, is a key determinant of the overall level of unsaturated FAs. Analyses of the ssi2 protein revealed that its substrate preference and regiospecificity were unaltered; however, its activity was 10-20 fold lower than that of the wt enzyme. Consistent with this finding, the 18:0 FA content was elevated in ssi2 plants, and the 16:3, 18:1 and 18:2 content was reduced. The composition of 16:0, 16:1, 16:2 and 18:3 in ssi2 plants was similar to or only slightly reduced from that of wt plants, presumably due to the activity of other S-ACP DES isoforms.

Since S-ACP DES catalyzes a desaturation step that is required for JA biosynthesis, we tested whether the induction of JA-dependent defense responses is affected in ssi2 plants. Both resistance to B. cinerea and induction of PDF1.2 expression were found to be impaired.

A likely explanation for these results is that activation of certain JA-dependent responses requires a second signal that is generated by S-ACP DES. Since ssi2 mutants would lack or have depressed levels of this co-activating signal, JA treatment would be insufficient to activate PDF1.2 expression or restore resistance to B. cinerea. By contrast, activation of strictly JA-dependent responses, such as THI2.1 and root growth inhibition, would remain unimpaired. Supporting this possibility is the discovery that injecting 18:1 into the leaves of ssi2 nahG plants restores YA-inducible PDF1.2 expression. The inability of 18:1 to rescue PDF1.2 expression in ssi2 plants is likely due to the high endogenous SA levels, which could antagonize JA's action. These results also suggest that 18:1 or an 181-derived signal works in conjunction with JA to induce JA-dependent defense gene expression and pathogen resistance.

In addition to lacking certain JA-induced defenses, ssi2 plants exhibit constitutive expression of several SA-associated defense responses. Since pathogen infection of wt plants gene induces the expression of either PDF1.2 or the PR genes, our results suggest that components of the FA desaturation pathway may cross regulate the activation of these defenses. Possibly, the co-activating signal inhibits the NPR1-independent pathway; loss of this signal in ssi2 plants would allow constitutive activation of the NPR1-independent responses (FIG. 11). Alternatively, the ratio of saturated versus unsaturated FAs or changes in their subcellular distribution might regulate cross-talk between defense signaling pathways. For example, an increase in 18:0 content might lead to activation of lipid signaling, which could then induce the PR signal transduction pathway (Anderson et al., 1998). Increases in unsaturated FAs also could stimulate (Klumpp et al., 1998) or inhibit (Baudouin et al., 1999) protein phosphatase(s) activity, which might then alter protein kinase- or mitogen activated protein kinase (MAPK)-regulated pathway(s), respectively. Interestingly, an Arabidopsis mutant defective in the MAPK mpk4 exhibits a phenotype similar to that of ssi2, including constitutive PR gene expression and suppressed PDF1.2 expression (Petersen et al., 2000). Perhaps reduced or altered unsaturated FA levels in the ssi2 mutant relieve inhibition of phosphatase activity which then results in inhibition of a MAPK (MPK4) pathway that negatively controls SA signaling and positively regulates JA signaling. The possibility that a decrease in S-ACP DES activity simply causes SA-mediated stress and PR gene expression is ruled out because the ssi2 phenotypes were seen in ssi2 nahG plants Example 1). Likewise, the possibility that stress due to high FA levels induces constitutive PR gene expression seems unlikely because fad2 mutants, which accumulate elevated levels of 18:1 (Miquel et al., 1992) and fad5 and fab1 mutants, which contain high levels of 16:0 (Ohlrogge & Browse, 1995), do not show any of the phenotypes displayed by ssi2 plants (data not shown). Furthermore, exogenous application of 18:0 does not induce PR-1 gene expression in wt plants (data not shown).

### REFERENCES CITED IN THE SPECIFICATION

Anderson, M. D., Chen, Z. & Klessig, D. F. (1998) Phytochemistry 47, 555-566.

Baudouin, E., Meskiene, I. & Hirt, H. (1999) Plant J. 20, 343-348.

Bell, C. J. & Ecker, J. R. (1994) Genomics 19, 137-144.

Bowling, S. A., Guo, A., Cao, H., Gordon, A. S., Klessig, D. F., and Dong, X. (1994) Plant Cell 6, 1845-1857.

Bowling, S. A., Clarke, J. D., Liu, Y., Klessig, D. F., and Dong, X. (1997) Plant Cell 9, 1573-1584.

Cao, H., Glazebrook, J., Clarke, J. D., Volko, S. & Dong, X. (1997) Cell 88, 57-63.

Clarke, J. D., Liu, Y., Klessig, D. F., and Dong, X. (1998) Plant Cell 10, 557-569.

Clough, S. J. & Bent, A. F. (1998) Plant J. 16, 735-743.

Dangl, J. L., Dietrich, R A., and Richberg, M. H. (1996) Plant Cell 8, 1793-1807.

Dietrich, R A., Delaney, T. P., Uknes, S. J., Ward, E. R, Ryals, J. A., and Dangl, J. L. (1994) Cell 77, 565-577.

Farmer, E. E. & Ryan, C. A. (1992) Plant Cell 4, 129-134.

Glazebrook, J., Rogers, E. E. & Ausubel, F. M. (1996) Genetics 143, 973-982.

Greenberg, J. T., Guo, A., Klessig, D. F., and Ausubel, F. M. (1994) Cell 7, 551-563.

Hamilton, C. M. (1997) Gene 200, 107-116.

Hunt, M. D., Delaney, T. P., Dietrich, R. A., Weymann, K. B., Dangl. J. L., Ryals, J. A. (1997) Mol. Plant-Microbe Interact. 10, 531-536.

Kachroo, P., Yoshioka, K., Shah, J., Dooner, H., and Klessig, D. F. (2000) Plant Cell 12,677-690.

Klumpp, S., Selke, D. & Hermesmeier, J. (1998) FEBS Lett. 437, 229-Konieczny, A & Ausubel, M. F. (1993) Plant J. 4, 403-410.

Lawton, K. A., Potter, S. L., Uknes, S. & Ryals, J. (1994) Plant Cell 6, 581-588.

Li, X., Zhang, Y., Clarke, J. D., Li, Y., and Dong, X. (1999) Cell 98, 329-339.

Liu, Y. G., Shirano, Y., Fukaki, H., Yanai, Y., Tasaka, M., Tabata, S. & Shibata, D. (1999) Proc. Natl. Acad. Sci. U.S.A. 96, 6535-6540.

Maleck, K. & Dietrich, R. A. (1999) Trends Plant Sci. 4,215-219.

Miquel, M. & Browse, J. (1992) J. Biol. Chem 267, 1502-1509.

Moore, I., Galweiler, L., Grosskopf, D., Schell, J. & Palme, K. (1998) Proc. Natl. Acad. Sci. U.S.A 95, 376-381.

Neff, M. M., Neff, J. D., Chory, J. & Pepper, A. E. (1998) Plant J. 14, 387-392.

Ohlrogge, J. & Browse, J. (1995) Plant Cell 7, 957-970.

Petersen, M., Brodersen, P., Naested, H., Andreasson, E., Lindhart, U., Johansen, B., Nielsen, H. B., Lacy, M., Austin, M. J., Parker, J. E., Sharma, S. B., Klessig, D. F., Martienssen, R., Mattsson, O., Jensen, A. B. & Mundy, J. (2000) Cell 103, 1111-1120.

Pieterse, C. M. J., Van Wees, S. C. M., Hoffland, E., Van Pelt, J. A., and Van Loon, L. C. (1996) Plant Cell 8, 1225-1237.

Pieterse, C. M. J., Van Wees, S. C. M., Van Pelt, J. A., Knoester, M., Laan, R., Gerrits, H., Weisbeek, P. J., and Van Loon, L. C. (1998) Plant Cell 10, 1571-1580.

Pieterse, C. M. J. & Van Loon, L. C. (1999) Trends Plant Sci. 4, 52-57.

Penninckx, I. A. M. A., Eggermont, K., Terras, F. R. G., Thomma, B. P. H. J., De Samblanz, G. W., Buchala, A., Métraux, J. -P., Manners, J. M. & Broekaert, W. F. (1996) Plant Cell 8, 2309-2323.

Rate, D. N., Cuenca, J. V., Bowman, G. R., Guttman, D. S. and Greenberg, J. T. (1999) Plant Cell 11, 1695-1708.

Ryals, J. A., Weymann, K., Lawton, K. Friedrich, L., Ellis, D., Steiner, H. -Y., Johnson, J., Delaney, T. P., Jesse, T., Voss, P. & Uknes, S. (1997) Plant Cell 9, 425-439.

Shah, J., Kachroo, P., and Klessig, D. F. (1999) Plant Cell 11, 191-206.

Shah, J., Tsui, F., and Kiessig, D. F. (1997) Mol. Plant-Microbe Interact. 10, 69-78.

Silva, H., Yoshioka, K, Dooner, H. K., and Klessig, D. F. (1999) Mol. Plant-Microbe. Interact. 10, 69-78.

Weymann, K., Hunt, M., Uknes, S., Neuenschwander, U., Lawton, K., Steiner, H. -Y., and Ryals, J. (1995) Plant Cell 7, 2013-2022.

Xiang, C., Han, P., Lutziger, I., Wang, K & Oliver, D. J. (1999) Plant Mol. Biol. 40, 711-717.

Yang, Y., Shah, J. & Klessig, D. F. (1997) Genes Dev. 11, 1621-1639.

Yu, I. C., Parker, J., and Bent, A. F. (1998) Proc. Natl. Acad. Sci. 95, 7819-7824.

This invention is not limited to the embodiments described and exemplified above, but is capable of variation and modification within the scope of the appended claims.

We claim:

1. An isolated nucleic acid molecule, comprising an SSI2 gene isolated from Arabidopsis thaliana chromosome 2 at a location within 0.2 cM from marker AthB102 and 3.7 cM from marker GBF, the disruption of which is associated with altered resistance of a plant to plant pathogens or other disease-causing agents.

2. The nucleic acid molecule of claim 1, wherein disruption of the gene in a plant causes the plant to exhibit a phenotype comprising one or more features selected from the group consisting of:
a) NPR1- and SA-independent constitutive expression of PR genes;
b) impairment of jasmonic acid-mediated activation of PDF1.2; and
c) accumulation of 18:0 fatty acids and decrease in 18:1 fatty acids.

3. The nucleic acid molecule of claim 1, which encodes a fatty acid desaturase.

4. The nucleic acid molecule of claim 3, wherein the encoded fatty acid desaturase is a Δ⁹ fatty acid desaturase.

5. The nucleic acid molecule of claim 4, wherein the Δ⁹ fatty acid desaturase possesses a substrate preference for 18:0 fatty acids.

6. A cDNA produced by reverse transcription of an mRNA encoded by the nucleic acid molecule of claim 1.

7. The nucleic acid molecule of claim 1, which encodes a polypeptide having greater than 60% identity to SEQ ID NO:3.

8. The nucleic acid molecule of claim 7, which encodes a polypeptide having SEQ ID NO:3.

9. The nucleic acid molecule of claim 8, comprising a coding sequence of SEQ ID NO:1 or SEQ ID NO:2.

10. An isolated nucleic acid molecule comprising a homolog of the nucleic acid molecule of claim 1, isolated from another plant species and encoding a Δ⁹ fatty acid desaturase.

11. The nucleic acid molecule of claim 10, the coding region of which is greater than 60% homologous to the coding region of SEQ ID NO:1 or SEQ ID NO:2.

12. A plant gene encoding a Δ⁹ fatty acid desaturse, wherein the coding region of the gene comprises a sequence selected from the group consisting of:
a) a coding region of SEQ ID NO:1 or SEQ D NO:2;
b) a sequence that hybridizes under stringent hybridization conditions with SEQ ID NO:1 or SEQ ID NO:2; and
c) a sequence encoding a Δ⁹ fatty acid desaturase having an amino acid sequence at least 60% identical to SEQ ID NO:3.

13. An isolated plant enzyme comprising a Δ⁹ fatty acid desaturase, wherein loss of function of the enzyme in a plant results in altered resistance of the plant to plant pathogens or other disease-causing agents.

14. The enzyme of claim 13, wherein loss of function of the enzyme in a plant causes the plant to exhibit one or more features selected from the group consisting of:
a) NPR1- and SA-independent constitutive expression of PR genes;
b) impairment of jasmonic acid-mediated activation of PDF1.2; and
c) accumulation of 18:0 fatty acids and decrease in 18:1 fatty acids.

15. The enzyme of claim 12, wherein the Δ⁹ fatty acid desaturase possesses a substrate preference for 18:0 fatty acids.

16. The enzyme of claim 15, which produces at least one product that functions in the plant as a defense response signal molecule or a precursor of a defense response signal molecule.

17. The enzyme of claim 16, wherein the defense response signal molecule is an 18:1 fatty acid or derivative thereof.

18. The enzyme of claim 16, wherein the defense response signal molecule inhibits a SA-independent defense response and participates in activation of a jasmonic-acid mediated defense response selected from the group consisting of activation of PDF1.2, resistance to Alternaria brassicicola and resistance to Botiytis citerea.

19. The enzyme of claim 18, wherein the defense response signal molecule inhibits an NPR1-independent defense response.

20. A plant-derived defense response signal molecule, produced directly or indirectly by activity of the enzyme of claim 12.

21. The defense response signal molecule of claim 20, comprising an 18:1 fatty acid or derivative thereof.

22. Antibodies immunologically specific for one or more epitopes of the enzyme of claim 12.

23. A mutant plant displaying a phenotype characterized by one or more features selected from the group consisting of:
a) NPR1- and SA-independent constitutive expression of PR genes;
b) impairment of jasmonic acid-mediated activation of PDF1.2; and
c) accumulation of 18:0 fatty acids and decrease in 18:1 fatty acids.

24. The mutant plant of claim 23, wherein the phenotype is conferred by a loss-of-function mutation in an SSI2 gene.

25. The mutant plant of claim 24, mutagenized by a method selected from the group consisting of chemical mutagenesis, radiation mutagenesis, and transposon or T-DNA insertions.

26. A method to enhance resistance of a plant to plant pathogens or other disease causing agents, comprising reducing or preventing function of a SSI2 gene product in the plant.

27. The method of claim 26, wherein the method results in a plant having features selected from the group consisting of:
a) NPR1- and SA-independent constitutive expression of PR genes;
b) impairment of jasmonic acid-mediated activation of PDF1.2; and
c) accumulation of 18:0 fatty acids and decrease in 18:1 fatty acids.

28. The method of claim 26, wherein the SSI2 function is reduced or prevented by the addition of at least one transgene to the plant genome.

29. The method of claim 28, wherein the transgene is the nucleic acid molecule of claim 12.

30. The method of claim 28, wherein the transgene is mutated and expression of the transgene produces a non-functional protein.

31. The method of claim 28, wherein the transgene expresses part or all of an antisense strand of the nucleic acid molecule of claim 12.

32. The method of claim 28, wherein an additional transgene is added to the plant genome, which expresses a sense strand of the nucleic acid molecule of claim 12, and addition of the transgene results in co-suppression of genes encoding the SSI2 gene product.

33. A fertile plant produced by the method of claim 26.

34. A method to enhance resistance of a plant to plant pathogens or other disease causing agents, comprising increasing production or activity of a SSI2 gene product in the plant.

35. The method of claim 34, wherein the enhanced resistance results from increased activity of jasmonic acid-mediated defense responses.

36. The method of claim 34, wherein the SSI2 production or activity is increased by the addition of at least one transgene to the plant genome.

37. The method of claim 36, wherein the transgene is the nucleic acid molecule of claim 12.

38. A fertile plant produced by the method of claim 34.
